(19) United States
(12) Patent Application Publication
(10) Pub. No.: US 2022/0211658 A1
(43) Pub. Date: Jul. 7, 2022

(54) METHODS FOR TREATING DISORDERS ASSOCIATED WITH SLEEP SPINDLE DEFICITS

(71) Applicants: The Broad Institute, Inc., Cambridge, MA (US); Massachusetts Institute of Technology, Cambridge, MA (US)

(72) Inventors: Zhanyan Fu, Cambridge, MA (US); Jennifer Q. Pan, Cambridge, MA (US); Ayan Ghoshal, Cambridge, MA (US); Guoping Feng, Cambridge, MA (US); Edward Scolnick, Cambridge, MA (US)

(73) Assignees: The Broad Institute, Inc., Cambridge, MA (US); Massachusetts Institute of Technology, Cambridge, MA (US)

(21) Appl. No.: 17/610,130

(22) PCT Filed: May 8, 2020
(86) PCT No.: PCT/US2020/032201

§ 371 Date: Nov. 9, 2021

Related U.S. Application Data
(60) Provisional application No. 62/846,530, filed on May 10, 2019.

(51) Int. Cl. A61K 31/343; A61K 31/381; A61K 45/06; A61P 25/20

(52) U.S. Cl. CPC A61K 31/343; A61P 25/20; A61K 45/06; A61K 31/381

(57) ABSTRACT

Provided herein are methods for treating a disorder associated with a sleep spindle deficit using a group II metabotropic glutamate receptor modulator.

### CROSS REFERENCE TO RELATED APPLICATIONS

This application claims the benefit under 35 U.S.C. § 119(e) of U.S. Provisional Application No. 62/846,530, filed May 10, 2019, entitled “Methods for Treating Disorders Associated With Sleep Spindle Deficits,” the entire disclosure of which is hereby incorporated by reference.

### FEDERALLY SPONSORED RESEARCH

This invention was made with government support under Grant No. MH115045, awarded by the National Institutes of Health. The government has certain rights in the invention.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: B119570063WO00-SEQ-OMJ.txt, date recorded: May 8, 2020; file size: 39 kilobytes).

### FIELD OF THE INVENTION

The present disclosure relates to treatment of disorders associated with a sleep spindle deficit by administering an effective amount of a pharmaceutical composition comprising a group II metabotropic glutamate receptor modulator.

### BACKGROUND

Sleep spindle oscillations are characteristic transient features of the sleep electroencephalogram (EEG). Although the underlying neural circuitry of sleep spindles has been relatively well characterized—being generated in the thalamic reticular nucleus and synchronized by thalamocortical interactions—their function is less clearly defined. Sleep spindles have an emerging role in memory and learning, synaptic plasticity, and neuropsychiatric disease.

### SUMMARY

The present disclosure is based, at least in part, on the finding that modulation of group II metabotropic glutamate receptors can alter sleep spindles. The present disclosure provides a therapeutic strategy for novel treatments of a disorder associated with a sleep spindle deficit.

Aspects of the disclosure relate to methods for treating a disorder associated with a sleep spindle deficit. Some aspects of the disclosure provide administering to a subject in need thereof an effective amount of a group II metabotropic glutamate receptor modulator. In some embodiments, the subject in need thereof has been diagnosed with a disorder associated with a sleep spindle deficit. In some embodiments, the subject has a mutation at the amino acid residue corresponding to amino acid residue 1346 in the CACNA1I protein depicted by SEQ ID NO: 1. Some aspects of the present disclosure provide determining that a subject has, is suspected of having, or is at risk of developing a disorder associated with a sleep spindle deficit. Some aspects of the present disclosure provide administering an effective amount of a group II metabotropic glutamate receptor modulator to a subject having, suspected of having, or at risk of developing, a disorder associated with a sleep spindle deficit.

In some embodiments, determining that a subject has, is suspected of having, or is at risk of developing a disorder associated with a sleep spindle deficit comprises detecting sleep spindles with an electroencephalogram (EEG). In some embodiments, determining that a subject has, is suspected of having, or is at risk of developing a disorder associated with a sleep spindle deficit comprises determining whether the subject has a mutation in the gene encoding the CACNA1I protein.

The present disclosure provides methods for treating a CACNA1I gene disorder in a subject. Some aspects of the present disclosure provide determining whether a subject has a mutation at the amino acid residue corresponding to amino acid residue 1346 in the CACNA1I protein depicted by SEQ ID NO: 1. Some aspects of the present disclosure provide administering an effective amount of a group II metabotropic glutamate receptor modulator to the subject. In some embodiments, the subject has a mutation at the amino acid residue corresponding to amino acid residue 1346 in the CACNA1I protein depicted by SEQ ID NO: 1. In some embodiments, the mutation at the amino acid residue corresponding to amino acid residue 1346 in the CACNA1I protein depicted by SEQ ID NO: 1 is a substitution of arginine to histidine. In some embodiments, the mutation at the amino acid residue corresponding to amino acid residue 1346 in the CACNA1I protein depicted by SEQ ID NO: 1 is a substitution of R1346H. In some embodiments, the CACNA1I gene disorder is associated with a sleep spindle deficit.

In some embodiments, the group II metabotropic glutamate receptor modulator is a mGluR_{2/3} agonist. In some embodiments, the mGluR_{2/3} agonist is LY354740. In some embodiments, the mGluR_{2/3} agonist is MGS0028. In some embodiments, the mGlUR_{2/3} agonist is LY379268. In some embodiments, the mGluR_{2/3} agonist is LY2934747. In some embodiments, the mGluR_{2/3} agonist is LY2969822. In some embodiments, the mGluR_{2/3} agonist is LY404040. In some embodiments, the mGluR_{2/3} agonist is LY404039. In some embodiments, the mGluR_{2/3} agonist is LY2140023.

In some embodiments, the group II metabotropic glutamate receptor modulator is a mGlu2-specific modulator. In some embodiments, the mGlu2-specific modulator is a mGlu2 agonist. In some embodiments, the mGlu2-specific modulator is a mGlu2 positive allosteric modulator (PAM). In some embodiments, the mGlu2 PAM is AZD8529. In some embodiments, the mGlu2 PAM is ADX-71149. In some embodiments, the mGlu2 PAM is JNJ-42153605. In some embodiments, the mGlu2 PAM is JNJ-40068782. In some embodiments, the mGlu2 PAM is GSK1331258. In some embodiments, the mGlu2 PAM is SAR218645. In some embodiments, the mGlu2 PAM is TASP0433864. In some embodiments, the mGlu2 PAM is LY487379. In some embodiments, the mGlu2 PAM is BINA.

In some embodiments, the group II metabotropic glutamate receptor modulator is a mGlu3-specific modulator. In some embodiments, the mGlu3-specific modulator is a mGlu3 agonist. In some embodiments, the mGlu3-specific modulator is a mGlu3 positive allosteric modulator (PAM). In some embodiments, the mGlu3 PAM is DT011088. In some embodiments, the mGlu3 PAM is Mavalon-63 PAM. In some embodiments, the mGlu3 PAM is Mavallon-207 PAM.

In some embodiments, the subject is a human subject.

In some embodiments, the subject is a human subject having, suspected of having, or at risk of developing a schizophrenia disorder. In some embodiments, the subject is a human subject having a mutation in the gene encoding the CACNA1I protein.

In some embodiments, methods described herein further comprise administering to the subject an additional therapeutic agent. In some embodiments, the additional therapeutic agent comprises one or more of aripiprazole, asenapine, brexpiprazole, buspirone, cariprazine, chlorpromazine hydrochloride, clozapine, haloperidol, iloperidone, loxapine, lumateperone, lurasidone hydrochloride, molindone hydrochloride, olanzapine, paliperidone, perphenazine, prochlorperazine, quetiapine, risperidone, thiothixene, trifluoperazine, ziprasidone, antipsychotics including aripiprazole, asenapine, cariprazine, loxapine, lurasidone hydrochloride, olanzapine, olanzapine and fluoxetine, quetiapine, risperidone, ziprasidone, clozapine, paliperidone, cariprazine, lurasidone, haloperidol, and chlorpromazine, antidepressants including fluoxetine, SSRIs including citalopram, escitalopram, paroxetine, and sertraline, SNRIs including desvenlafaxine, duloxetine, and venlafaxine, tricyclics including amitriptyline, desipramine, imipramine, and nortriptyline, and MAOIs including phenelzine and tranylcypromine, anticonvulsants including carbamazepine, divalproex sodium, lamotrigine, valproate sodium, valproic acid, and topiramate, mood stabilizers including lithium and lithium carbonate, benzodiazepines including lorazepam, clonazepam, diazepam, alprazolam, and chlordiazepoxide, metformin, memantine, flumazenil, or meclofenoxate, risperidone, lithium carbonate, methylphenidate, procyclidine, ferrous futnarate+vitamins+lactulose+cod liver oil+various skin ointments, clobazam+lorazepam, rectal diazepam+buccal midazolam, ethosuximide, felbamate, gabapentin, gabapentin and lidocaine, gabapentin and lidocaine and prilocaine, lacosamide, levertiracetam, oxcarbazepine, perampanel, topiramate, valproate, zonisamide, cannabidiol, cenobamate, phenytoin, ezogabine, rufinamide, stiripentol, vigabatrin, eslicarbazepine acetate, pregabalin, and tiagabine, midazolam, clobazam, barbiturates including phenobarbital and primidone, stimulants including methylphenidate, or alpha-2-adrenergic agonists including clonidine, and guanfacine.

In some embodiments, a disorder associated with a sleep spindle deficit is associated with a schizophrenia disorder in a subject. In some embodiments, the disorder associated with the sleep spindle deficit is a schizophrenia disorder. In some embodiments, the disorder is bipolar disorder. In some embodiments, the disorder is intellectual disability. In some embodiments, the disorder is schizophrenia. In some embodiments, the disorder is epilepsy. In some embodiments, the disorder is an autism spectrum disorder.

In some embodiments, the schizophrenia disorder is paranoid schizophrenia. In some embodiments, the schizophrenia disorder is disorganized schizophrenia. In some embodiments, the schizophrenia disorder is catatonic schizophrenia. In some embodiments, the schizophrenia disorder is childhood schizophrenia. In some embodiments, the schizophrenia disorder is schizoaffective disorder.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations of thereof herein, is meant to encompass the items listed thereafter and equivalents thereof as well as additional items.

### BRIEF DESCRIPTION OF THE DRAWINGS

The accompanying drawings are not intended to be drawn to scale. The drawings are illustrative only and are not required for enablement of the disclosure. For purposes of clarity, not every component may be labeled in every drawing. In the drawings:

FIG. 1A shows representative traces of the rebound burst firing in a TRN neuron in WT mice before (upper panel) and after (lower panel) the bath application of the group II metabotropic glutamate receptor agonist LY379268.

FIG. 1B shows a graph summarizing the maximum effect of the LY379268 on the number of rebound bursts among individual WT TRN neurons, plotting number of rebound bursts observed before (baseline) and after the treatment with LY379268 (n=15 recordings; Wilcoxon signed rank test: p=0.002**). Such effects were not observed with ACSF treatment (n=14 recordings; Wilcoxon signed rank test: p=0.1562).

FIG. 1C shows a graph summarizing the maximum effect of the LY379268 on the threshold of rebound bursts among individual WT TRN neurons, plotting the burst threshold before (baseline) and after the treatment with LY379268 (n=10 recordings; Wilcoxon signed rank test: p=0.048*). Such effects were not observed with ACSF treatment (n=10 recordings; Wilcoxon signed rank test: p=0.3750).

FIG. 2A shows representative traces of the rebound burst firing in a TRN neuron in RH mice before (upper panel) and after (lower panel) the bath application of the group II metabotropic glutamate receptor agonist LY379268.

FIG. 2B shows a graph summarizing the maximum effect of the LY379268 on the number of rebound bursting among individual RH TRN neurons, plotting number of rebound bursts observed before (baseline) and after the treatment with LY379268 (n=9 recordings; Wilcoxon signed rank test: p=0.0039**). Such effects were not observed with ACSF treatment (n=10 recordings; Wilcoxon signed rank test: p=0.7500).

FIG. 3A shows representative traces of the rebound burst firing in a TRN neuron in WT mice before (upper panel) and after (lower panel) the bath application of the group II metabotropic glutamate receptor agonist LY404039.

FIG. 3B shows a graph summarizing the maximum effect of the LY404039 on the number of rebound bursting among WT TRN neurons, plotting number of rebound bursts observed before (control) and after the treatment with LY404039 (n=7 recordings; Wilcoxon signed rank test: p=0.0156*).

FIG. 4A shows representative traces of the rebound burst firing in a TRN neuron in WT mice before (upper panel) and after (lower panel) the bath application of the group II metabotropic glutamate receptor positive allosteric modulator (PAM) Mavalon-63 (Mav63).

FIG. 4B shows a graph summarizing the maximum effect of the Mav63 on the number of rebound bursting among the individual WT TRN neurons, plotting number of rebound bursts observed before (control) and after the treatment with Mav63 (n=12 recordings; Wilcoxon signed rank test: p=0.0020**).

FIG. 5A is a schematic depiction of the experimental design to test the effect of 10 mg/kg LY379268 administration on sleep spindles. 12 hour light cycles for each condition were analyzed for NREM sleep duration, relative delta power, and sleep spindle density.

FIG. 5B is a graph showing percentage of time spent in NREM sleep that was observed in both WT and RH mice (n=8 mice) following the administration of LY379268. *signifies p<0.05 (paired t-test).

FIG. 5C is a graph showing relative delta power in both WT and RH mice that was observed following administration of LY379268 or 0.9% saline (vehicle). * signifies p<0.05 compared to WT baseline; # signifies p<0.05 compared to RH baseline, respectively (Dunnett's multiple comparison test; post-hoc to 2 way Mixed effect model analysis).

FIG. 5D is a graph showing spindle density for the entire 12 hour light cycle NREM sleep that was calculated for spindle oscillations with center spindle frequencies (Fc) of 13 Hz (left panel) and 15 Hz (right panel). * signifies p<0.05 compared to WT baseline; # and ## signifies p<0.05 and p<0.01 compared to RH baseline (Dunnett's multiple comparison test; post-hoc to 2 way Mixed-effects model analysis)

FIG. 5E is a graph showing spindle densities that were calculated for the last 6 hours of the 12 hour light cycle NREM sleep for both Fc=13 Hz (left panel) and 15 Hz spindles (right panel). * and ** signifies p<0.05 and p<0.01 respectively compared to WT baseline; ### signifies p<0.001 compared to RH baseline (Dunnett's multiple comparison test; post-hoc to 2 way Mixed-effects model analysis).

FIG. 6A is a schematic depiction of the experimental design to test the effect of 10 mg/kg Mav63 administration on sleep spindles. 12 hour light cycles for each condition were analyzed for sleep spindle density.

FIG. 6B is a graph showing spindle density for the entire 12 hour light cycle NREM sleep that was calculated for spindle oscillations with spindle frequencies 9-16 Hz. * signifies p<0.05 (Paired t-test).

### DETAILED DESCRIPTION

Sleep spindles are rhythms that occur periodically during non-rapid eye movement (NREM) sleep, and are associated with oscillatory discharges of neurons throughout the thalamocortical system and are thought to be generated in the thalamic reticular nucleus (TRN). Disruption of sleep spindle activity is associated with disorders such as schizophrenic disorders. The present disclosure is based, at least in part, on the finding that mice having a mutation in the schizophrenia risk gene CACNA1I exhibited disrupted channel function, altered thalamic excitability and abnormal sleep spindles that could be rescued by administering a group II metabotropic glutamate receptor modulator. Accordingly, the present disclosure provides methods for treating a subject having a disorder associated with a sleep spindle deficit using an effective amount of a group II metabotropic glutamate receptor modulator.

### Disorders Associated with a Sleep Spindle Deficit

As used herein, “sleep spindles” refers to bursts of oscillatory brain activity generated in the reticular nucleus of the thalamus that occur during sleep. In some embodiments, sleep spindles comprise a burst of 12-15 Hz. In some embodiments, sleep spindles occur during stage 2 non-rapid eye movement (NREM) sleep (N2). In some embodiments, sleep spindles occur during stage 3 non-rapid eye movement (NREM) sleep (N3).

As used herein, a “sleep spindle deficit” refers to a deficit in any aspect of a sleep spindle, including, but not limited to, number of spindles, spindle density, spindle amplitude, spindle duration, spindle peak frequency, and spindle sigma power.

A “disorder associated with a sleep spindle deficit,” as used herein, refers to any disorder associated with a deficit in a subject's sleep spindles or a deficit in one or more characteristics associated with a subject's sleep spindles. Characteristics of sleep spindles include but are not limited to: the total number of sleep spindle oscillations throughout the NREM sleep, sleep spindle density (e.g., number of sleep spindles per minute during sleep), sleep spindle duration, sleep spindle amplitude, sleep spindle frequency power, sleep spindle integrated power, sleep spindle morphology, spindle symmetry, and sleep spindle coherence with slow wave oscillations. Sleep spindle deficits include, but are not limited to, deviation in one or more of these characteristics from a population mean by more than 0.5 standard deviation (SD), 1 SD, or 2 SD. In some embodiments, sleep spindle deficits include, but are not limited to, deviation in one or more of these characteristics from a close relative by more than 0.5 SD, 1 SD, or 2 SD. Examples of disorders associated with a sleep spindle deficit include, but are not limited to, bipolar disorder, intellectual disability, schizophrenia disorders, epilepsy, and autism spectrum disorders. in some embodiments, disorders associated with a sleep spindle deficit are schizophrenia disorders. Schizophrenia disorders include, but are not limited to, paranoid schizophrenia, disorganized schizophrenia, catatonic schizophrenia, childhood schizophrenia, and schizoaffective disorder. It should be appreciated that any disorder associated with a sleep spindle deficit, including any schizophrenia disorder, is encompassed by aspects of the disclosure.

### Detecting Sleep Spindles

Methods described herein encompass detecting a sleep spindle deficit. In some embodiments, a sleep spindle deficit comprises a deficit, relative to a control, in any aspect of a sleep spindle, of at least 5%, 10%, 20%, 30%, 40%, 50%, 60%, 70%, 80%, 90%, 100%, or at least 2-fold, at least 5-fold, at least 10-fold, at least 20-fold, at least 50-fold, or at least 100-fold. In some embodiments, a sleep spindle deficit comprises a deficit in at least one aspect of a sleep spindle. In some embodiments, a sleep spindle deficit comprises a deficit in at least two, at least three, at least four, at least five, at least six, at least seven, at least eight, at least nine, or at least ten aspects of a sleep spindle. In some embodiments, a sleep spindle deficit comprises a deficit in more than one aspect of a sleep spindle.

In some embodiments, methods described herein comprise analysis of sleep spindle data. As used herein, “sleep spindle data” refers to any data related to a subject's sleep spindles. Detection of sleep spindle data can involve use of an electroencephalogram (EEG) and/or a magnetoencephalogram (MEG). In some embodiments, detection of sleep spindle data can involve use of polysomnography (PSG). It should be appreciated that any method known to one of ordinary skill in the art for detecting sleep spindles can be compatible with aspects of the disclosure.

In some embodiments, processing sleep spindle data comprises filtering. In some embodiments, the filtering comprises filtered N2 sleep. In some embodiments, filtering comprises band-pass filtering. In some embodiments, filtering comprises Buckelmuiller filtering. In some embodiments, filtering comprises RMS filtering. In some embodiments, filtering comprises Hjorth filtering. In some embodiments, processing sleep spindle data comprises artifact suppression. In some embodiments, the artifact suppression is ECG artifact suppression. In some embodiments, processing sleep spindle data comprises spectral analysis. In some embodiments, processing sleep spindle data comprises spindle detection. In some embodiments, spindle detection comprises wavelet method or sigma bandpass method. In some embodiments, processing sleep spindle data comprises phenotype compilation. In some embodiments, processing sleep spindle data comprises visual inspection.

In some embodiments, processing sleep spindle data comprises the use of software suitable for determining the presence of sleep spindles. One of ordinary skill in the art would be familiar with software that is compatible with aspects of the present disclosure. For example, Luna (zzz.bwh.harvard.edu, Ghoshal et al, 2020) can be used for processing sleep spindle data and determining the presence of sleep spindles. Luna (current release v0.23 (Jan. 15, 2020) is an open-source C/C++ software package for manipulating and analyzing polysomnographic recordings, with a focus on the sleep EEG. The Luna package comprises lunaC, a command-line interface to the Luna C/C++ library and lunaR, a package for the R statistical software. Various commands are available on Luna for detecting and processing sleep spindle data. For example, Luna comprises SPINDLES command, which can detect spindles using a wavelet-based approach. The SPINDLES command is able to detect slow oscillations (SO) and the temporal coupling between spindles and SO. A single SPINDLES command can detect spindles at different frequencies, and on different channels. In another example, Luna comprises the FILTER command, which applies a linear-phase FIR filter to a signal. The FILTER command modifies the in-memory signal by applying a finite impulse response (FIR) filter, which can be either a low-pass, high-pass, band-pass or band-stop filter. Aspects related to the use of Luna for analyzing sleep spindles can be found, at least, on zzz.bwh.harvard.edu/luna/, the entire contents of which are incorporated herein by reference.

In some embodiments, a deep learning strategy such as SpindleNet can be used for detecting sleep spindles based on a single EEG channel for real-time sleep spindle detection. Aspects related to the use of SpindleNet for analyzing sleep spindles can be found in Kulkarni et al., A deep learning approach for real-time detection of sleep spindles, J Neural Eng. 2019 June; 16(3): 036004. Doi: 10.1088/1741-2552/ab0933, the entire contents of which are incorporated herein by reference.

Other aspects related to processing sleep spindle data are found in Purcell et al., Characterizing sleep spindles in 11,630 individuals from the National Sleep Research Resource, Nature Comm., 2017; 8: 15930, and Manoach et al., “Reduced Sleep Spindles in Schizophrenia: A Treatable Endophenotype That Links Risk Genes to Impaired Cognition?” Biological Psychiatry 2016; 80:599-608, the entire contents of each of which are incorporated herein by reference.

In some embodiments, methods comprise obtaining sleep spindle data from a subject. In some embodiments, methods comprise obtaining sleep spindle data from a publicly available database. In some embodiments, sleep spindle data and other aspects of the data related to sleep spindles can be in the European Data Format (EDF). Any database containing information associated with sleep spindles may be used in methods described herein. Examples of databases include, but are not limited to, National Sleep Research Resource (NSRR), Childhood Adenotonsillectomy Trial (CHAT), Cleveland Children's Sleep and Health Study (CCSHS), Cleveland Family Study (CFS), Sleep Heart Health Study (SHHS), Outcomes of Sleep Disorders In Older Men Study (MrOS-Sleep), and Study of Osteoporotic Fractures (SOF).

### Group II Metabotropic Glutamate Receptor Modulators

The metabotropic glutamate receptors (mGluRs) are a family G-protein-coupled receptors that participate in the modulation of synaptic transmission and neuronal excitability throughout the central nervous system. The mGluRs bind glutamate within a large extracellular domain and transmit signals through the receptor protein to intracellular signaling partners. Genes encoding eight mGluR subtypes have been identified, many with multiple splice variants that are differentially expressed in distinct cell types throughout the CNS. mGluRs are subclassified into three groups based on sequence homology, G-protein coupling, and ligand selectivity. Group I includes mGluRs 1 and 5, Group II includes mGluRs 2 and 3, and Group in includes mGluRs, 4, 6, 7, and 8.

Aspects of the disclosure relate to treating a disorder associated with a sleep spindle deficit with group II metabotropic glutamate receptor modulators. In some embodiments, the group II metabotropic glutamate receptor modulator blocks or inhibits a biological response by binding to a group II metabotropic glutamate receptor (e.g., a group II metabotropic glutamate receptor antagonist). In other embodiments, the group II metabotropic glutamate receptor modulator binds to a group II metabotropic glutamate receptor and activates the receptor to produce a biological response (e.g., a group II metabotropic glutamate receptor agonist). In other embodiments, the group II metabotropic glutamate receptor modulator modulates a biological response of a group II metabotropic glutamate receptor allosterically by binding to a site different than the ligand binding site (e.g., a group II metabotropic glutamate receptor positive allosteric modulator (PAM) or a group II metabotropic glutamate receptor negative allosteric modulator (NAM)).

A group II metabotropic glutamate receptor modulator, as used herein, refers to any ligand that modulates (e.g., increases or decreases) a biological response of a group II metabotropic glutamate receptor. Group II metabotropic glutamate receptor modulators include group II metabotropic glutamate receptor 2 (mGlu₂) and group II metabotropic glutamate receptor 3 (mGlu₃) modulators.

In some embodiments, a group II metabotropic glutamate receptor modulator used in the methods described herein increases a biological response of a group II metabotropic glutamate receptor by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold. In some embodiments, a group II metabotropic glutamate receptor modulator used in the methods described herein decreases a biological response of a group II metabotropic glutamate receptor by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold.

Examples of group II metabotropic glutamate receptor modulators include, but are not limited to, group II metabotropic glutamate receptor agonists, group II metabotropic glutamate receptor antagonists, group II metabotropic glutamate receptor negative allosteric regulators (NAMs), and group II metabotropic glutamate receptor positive allosteric regulators (PAMs).

In some embodiments, the group II metabotropic glutamate receptor modulator interacts with both mGlu₂ and mGlu₃. In certain embodiments, the group II metabotropic glutamate receptor modulator interacts differently with mGlu₂ and mGlu₃. For example, the group II metabotropic glutamate receptor modulator may be a mGlu₂ agonist and a mGlu₃ antagonist (e.g., LY395756). In other embodiments, the group II metabotropic glutamate receptor modulator may be a mGlu₂ antagonist and a mGlu₃ agonist.

In some embodiments, the group II metabotropic glutamate receptor modulator specifically interacts with mGlu₂ or mGlu₃. Specific interaction of a ligand with a receptor, such as mGlu₂ or mGlu₃, is well-understood in the art, and methods to determine such specific interactions are also well-known in the art. A group II metabotropic glutamate receptor modulator is said to exhibit specific interaction with a receptor, such as mGlu₂ or mGlu3, if it reacts or associates more frequently, more rapidly, with greater duration, and/or with greater affinity with a receptor, such as mGlu₂ or mGlu₃, than it does with another receptor. It should also be understood that a ligand that specifically interacts with mGlu₂ or mGlu₃may or may not specifically or preferentially interact with another receptor (e.g., a group I mGlu receptor). As such, specific interaction or preferential interaction does not necessarily require (although it can include) exclusive binding or interaction.

In some embodiments, the group II metabotropic glutamate receptor modulator is a mGlu₂-specific modulator. Examples of mGlu₂-specific modulators include, but are not limited to, mGlu₂ agonists, mGlu₂ antagonists, mGlu₂NAMs, and mGlu₂ PAMs. In some embodiments, the group II metabotropic glutamate receptor modulator is a mGlu₃-specific modulator. Examples of mGlu₃-specific modulators include, but are not limited to, mGlu₃ agonists, mGlu₃ antagonists, mGlu₃NAMs, and mGlu₃PAMs.

In some embodiments, mGluR₃ is selectively enriched in the TRN. In some embodiments, the augmentation of mGluR₃ activity by an mGluR₃ PAM corrects the reduced rebound bursting of TRN neurons.

In some embodiments, the group II metabotropic glutamate receptor modulator is a mGluR_{2/3} agonist. In some embodiments, the mGluR_{2/3} agonist is selected from the group consisting of LY354740, MGS0028, LY379268, LY2934747, LY2969822, LY404040, LY404039, and LY2140023. In some embodiments, the mGluR_{2/3} agonist is LY379268. In some embodiments, the mGluR_{2/3} agonist is LY404039.

In some embodiments, the group II metabotropic glutamate receptor modulator is a mGluR_{2/3} antagonist. In some embodiments, the group II metabotropic glutamate receptor antagonist is selected from the group consisting of LY341495, LY3020371, HYDIA, MGS0039, and CECXG.

In some embodiments, the group II metabotropic glutamate receptor modulator is a mGluR_{2/3} NAM. In some embodiments, the mGluR_{2/3} NAM is RO4491533 or MNI-137. In some embodiments, the mGluR_{2/3} NAM is RO4491533. In some embodiments, the mGluR_{2/3} NAM is MNI-137.

In some embodiments, the group II metabotropic glutamate receptor modulator is a mGlu₂ PAM. In some embodiments, the mGlu₂ PAM is selected from the group consisting of AZD8529, ADX-71149, JNJ-42153605, JNJ-40068782, GSK1331258, SAR218645, TASP0433864, LY487379, and BINA.

In some embodiments, the group II metabotropic glutamate receptor modulator is a mGlu₂ NAM. In some embodiments, the mGlu₂ NAM is VU6001966.

In some embodiments, the group II metabotropic glutamate receptor modulator is a mGlu₂ agonist. In some embodiments, the mGlu₂ agonist is LY2812223 or LY2979165. In some embodiments, the mGlu₂ receptor agonist is LY2812223. In some embodiments, the mGlu₂ receptor agonist is LY2979165.

In some embodiments, the group II metabotropic glutamate receptor modulatoris a mGlu₃ agonist. In some embodiments, the mGlu₃ agonist is LY2794193.

In some embodiments, the group II metabotropic glutamate receptor modulator is a mGlu₃ PAM. In some embodiments, the mGlu₃ PAM is selected from the group consisting of DT011088, Mavalon-63 (Mav63) PAM, and Mavalon-207 (Mav207) PAM. In some embodiments, the mGlu₃ PAM is Mavalon-63 (Mav63).

In some embodiments, the group II metabotropic glutamate receptor modulator is a mGlu₃NAM. In some embodiments, the mGlu₃ NAM is selected from the group consisting of VU0650786, ML337, and LY2389575. In some embodiments, the group II metabotropic glutamate receptor modulator is a group II metabotropic glutamate receptor modulator disclosed in, and incorporated by reference from, US2015361081 or WO2016130652.

Examples of group II metabotropic glutamate receptor modulators and their structures are provided in Table 1.

TABLE 1
Non-limiting examples of group II metabotropic glutamate receptor modulators.

| Name | Chemical Name | Chemical Structure | Function | Reference |
|---|---|---|---|---|
| LY2794193 | (1S,2S,4S, 5R,6S)-2-amino- 4-[(3- methoxybenzoyl) amino]bicyclo [3.1.0]hexane-2,6- dicarboxylic acid | 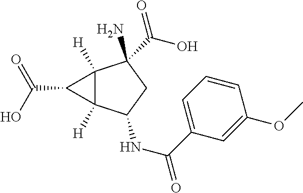 [SMILES: COc1cccc(C(=O)N[C@H]2C[C@@](N)(C(=O)O)[C@@H]3[C@@H](C(=O)O)[C@@H]32)c1] | mGlu₃ Agonist | Journal of Medicinal Chemistry (2018), 61(6), 2303-2328 |
| DT011088 | Not reported | Not reported | mGlu₃ | Domain |
| | | | PAM | Therapeutics |
| Mavalon 63 (Mav63) | 5-Methyl-9-(5- fluoropyridin-2-yl)- spiro[benzo [f]pyrrolo[1.2- a][1,4]diazepine-6,1′- cyclopropan]-4(5H)-one | 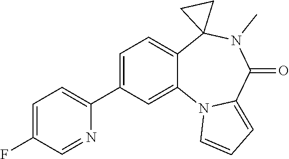 [SMILES: CN1C(=O)c2cccn2-c2cc(-c3ccc(F)cn3)ccc2C12CC2] | mGlu₃ PAM | WO2017081483A1 WO2018206820A1 |
| Mavalon 207 (Mav207) | 2-Methoxymethyl- 5-methyl- 9-(6-fluoro- pyridin-3-yl)- spiro[benzo[f] pyrazolo[1,5- a][1,4]diazepine-6.1′- cyclopropan]- 4(5H)-one | 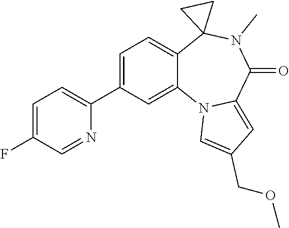 [SMILES: COCc1cc2n(c1)-c1cc(-c3ccc(F)cn3)ccc1C1(CC1)N(C)C2=O] | mGlu₃ PAM | WO2017081483A1 WO2018206820A1 |
| Merck example 1 | 1-isopropyl- 3-morpholin-4- yl-5,6,7,8- tetrahydroisoquinoline- 4-carbonitrile | 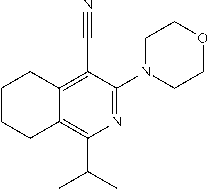 [SMILES: CC(C)c1nc(N2CCOCC2)c(C#N)c2c1CCCC2] | mGlu₃ PAM | WO2014117919A1 |
| VU0650786 | (R)-2-(((5- chloropyridin-2- yl)oxy)methyl)-5-(2- fluoropyridin-3-yl)-7- methyl-6,7- dihydropyrazolo[1,5- a]pyrazin-4(5H)-one | 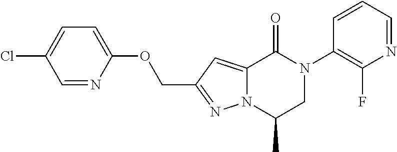 [SMILES: C[C@@H]1CN(c2cccnc2F)C(=O)c2cc(COc3ccc(Cl)cn3)nn21] | mGlu₃ NAM | Journal of Chemistry (2015), 58(18), 7485-7500 |
| ML337 | (R)-(2-fluoro-4-((-4- methoxyphenyl) ethynyl)phenyl) (3-hydroxypiperidin-1- yl)methanone | 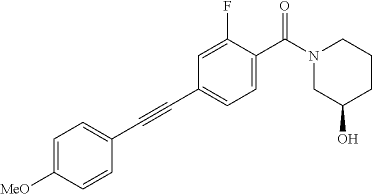 [SMILES: COc1ccc(C#Cc2ccc(C(=O)N3CCC[C@@H](O)C3)c(F)c2)cc1] | mGlu₃ NAM | Journal of Chemistry (2013), 56(12), 5208-5212 |
| LY2389575 | (3S)-N-(2,4- Dichlorobenzyl)-1-(5- bromopyrimidin-2- yl)pyrrolidinyl-3-amine | 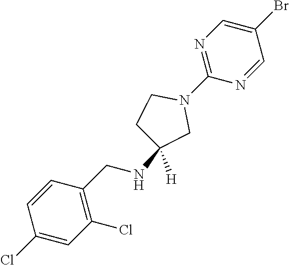 [SMILES: Clc1ccc(CN[C@H]2CCN(c3ncc(Br)cn3)C2)c(Cl)c1] | mGlu₃ NAM | WO2006044454A1 |
| US2015361081 Compound 37 | 2-(((4- (difluoromethoxy)-2- fluorophenyl) amino)methyl)- 5-(4-fluorophenyl)-6,7- dihydropyrazolo[1,5- a]pyrazin-4(5H)-one | 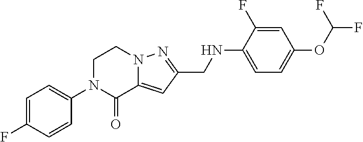 [SMILES: O=C1c2cc(CNc3ccc(OC(F)F)cc3F)nn2CCN1c1ccc(F)cc1] | mGlu₃ NAM | US2015361081 |
| WO2016130652 example 1 | 1-(4-Fluorophenyl)-4- hydroxypyridin- 2(1H)-one | 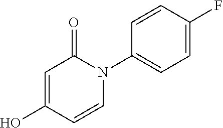 [SMILES: O=c1cc(O)ccn1-c1ccc(F)cc1] | mGlu₃ NAM | WO2016130652 |
| LY2812223 | (1R,2S,4R, 5R,6R)-2-amino- 4-(1H-l,2,4-triazol-3- ylsulfanyl)bicyclo[3.1.0] hexane-2,6-dicarboxylic acid | 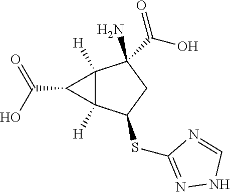 [SMILES: N[C@@]1(C(=O)O)C[C@@H](Sc2nc[nH]n2)[C@H]2[C@H](C(=O)O)[C@H]21] | mGlu₂ Agonist | US20110152334A1 |
| LY2979165 | (1S,2R,4S, 5S,6S)-4-((4H- l,2,4-triazol- 3-yl)thio)-2- ((R)-2- aminopropanamido) bicyclo [3.1.0]hexane-2,6- dicarboxylic acid | 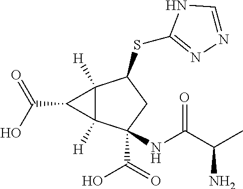 [SMILES: C[C@@H](N)C(=O)N[C@]1(C(=O)O)C[C@H](Sc2nnc[nH]2)[C@@H]2[C@@H](C(=O)O)[C@@H]21] | mGlu₂ Agonist | US20110152334A1 |
| AZD8529 | 1H-Isoindol-1-one, 2,3- dihydro-7- methyl-5-[3-(1- piperazinylmethyl)- 1,2,4- oxadiazol-5-yl]-2-[[4- (trifluoromethoxy) phenyl] methyl]- | 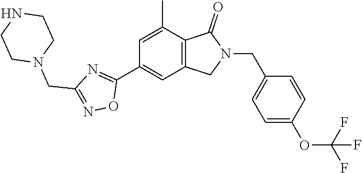 [SMILES: Cc1cc(-c2nc(CN3CCNCC3)no2)cc2c1C(=O)N(Cc1ccc(OC(F)(F)F)cc1)C2] | mGlu₂ PAM | US8377940B2 and WO2008150233A1 |
| ADX-71149 | 1-(4-Chloro-2- fluorobenzyl)-5-(4- methoxyphenyl)-2(1H)- pyridinone | 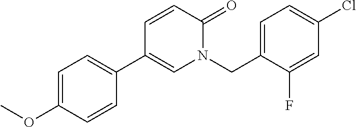 [SMILES: COc1ccc(-c2ccc(=O)n(Cc3ccc(Cl)cc3F)c2)cc1] | mGlu₂ PAM | WO2009033704A1 |
| JNJ-42153605 | 3-cyclopropylmethyl)- 7-(4- phenylpiperidin- 1-yl)-8- (trifluoromethyl)- [1,2,4]triazolo[4,3- a]pyridine | 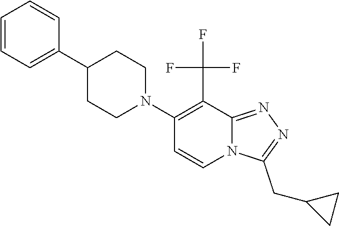 [SMILES: FC(F)(F)c1c(N2CCC(c3ccccc3)CC2)ccn2c(CC3CC3)nnc12] | mGlu₂ PAM | WO2010130424A1 |
| JNJ-40068782 | 3-Cyano-1- cyclopropylmethyl-4-(4- phenyl-piperidin-l-yl)- pyridine-2(1H)-one | 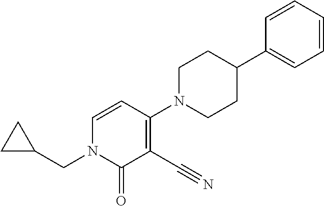 [SMILES: N#Cc1c(N2CCC(c3ccccc3)CC2)ccn(CC2CC2)c1=O] | mGlu₂ PAM | WO2008107479A1 and WO2007104783A2 |
| GSK1331258 | 2-[[4-[3-chloro-5- (trifluoromethyl) pyridin-2- yl]piperazin-1- yl]methyl]-1- methylbenzimidazole | 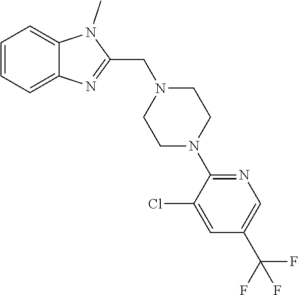 [SMILES: Cn1c(CN2CCN(c3ncc(C(F)(F)F)cc3Cl)CC2)nc2ccccc21] | mGlu₂ PAM | Bioorganic & Medicinal Chemistry Letters (2010), 20(2), 759- 762 |
| SAR218645 | ((S)-2-(1,1- dimethyl-indian- 5-yloxymethyl)-2,3- dihydro-oxazolo[3,2- a]pyrimidin-7-one) | 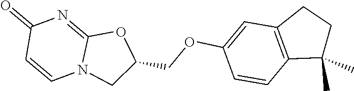 | mGlu₂ PAM | WO2011034830A1 |
| TASP0433864 | [(2S)-2-[(4-tert- butylphenoxy)methyl]-5- methyl-2,3- dihydroimidazo[2,1- b][1,3]oxazole-6- carboxamide] | 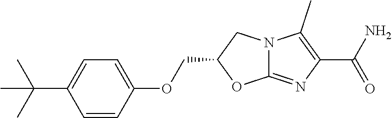 [SMILES: Cc1c(C(N)=O)nc2n1C[C@@H](COc1ccc(C(C)(C)C)cc1)O2] | mGlu₂ PAM | WO2013062079A1 |
| LY487379 | 2,2,2- Trifluoro-N-[4-(2- methoxypheoxy) phenyl]-N-(3- pyridinylmethyl) ethanesulfonamide | 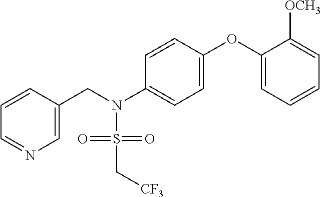 [SMILES: COc1ccccc1Oc1ccc(N(Cc2cccnc2)S(=O)(=O)CC(F)(F)F)cc1] | mGlu₂ PAM | WO2001056990A2 |
| BINA | 3′-[[(2-Cyclopentyl-2,3- dihydro-6,7-dimethyl-1- oxy]methyl]-[1,1′- biphenyl]-4- carboxylic acid | 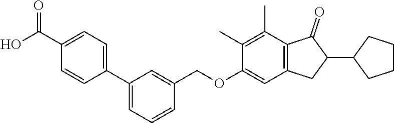 [SMILES: Cc1c(OCc2cccc(-c3ccc(C(=O)O)cc3)c2)cc2c(c1C)C(=O)C(C1CCCC1)C2] | mGlu₂ PAM | WO2006015158A1 |
| VU6001966 | 4-(4- fluorophenyl)-5-((1- methyl-1H-pyrazol-3- yl)methoxy) picolinamide | 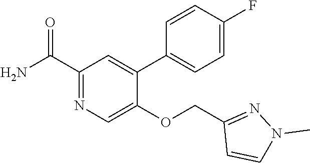 [SMILES: Cn1ccc(COc2cnc(C(N)=O)cc2-c2ccc(F)cc2)n1] | mGlu₂ PAM | WO2016149324A1 |
| LY354740 | (1S,2S,5R,6S)-2- aminobicyclo [3.1.0]hexane- 2,6-dicarboxylic acid | 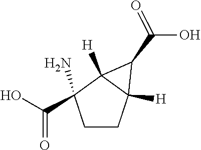 [SMILES: N[C@@]1(C(=O)O)CC[C@H]2[C@H](C(=O)O)[C@H]21] | mGlu₂ and mGlu₃ Agonist | EP696577A1 and US5882671 |
| MGS0028 | (1R,2S,5S,6S)- 2-amino-6- fluoro-4- oxobicyclo [3.1.0]hexane- 2,6-dicatoxylic acid | 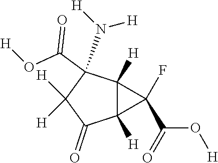 [SMILES: N[C@@]1(C(=O)O)CC(=O)[C@@H]2[C@H]1[C@@]2(F)C(=O)O] | mGlu₂ and mGlu₃ Agonist | WO2000012464A1 |
| LY379268 | (1R,4R,5S, 6R)-4-Amino-2- oxabicyclo[3.1.0]hexane- 4,6-dicarboxylic acid | 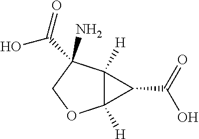 [SMILES: N[C@]1(C(=O)O)CO[C@@H]2[C@@H](C(=O)O)[C@@H]21] | mGlu₂ and mGlu₃ Agonist | US5688826 and EP774461 |
| LY2934747 | (1R,4S,5S,6S)-4- aminospiro [bicyclo[3.1.0] hexane-2,1′- cyclopropane]- 4,6-dicathoxylic acid | 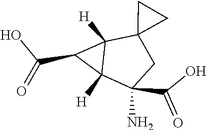 [SMILES: N[C@@]1(C(=O)O)CC2(CC2)[C@H]2[C@H](C(=O)O)[C@H]21] | mGlu₂ and mGlu₃ Agonist | US20130197079A1 |
| LY2969822 | (1R,4S,5S,6S)-4-((S)-2- aminopropanamido) spiro[bicyclo[3.1.0] hexane-2,1′- cyclopropane]- 4,6-dicarboxylic acid | 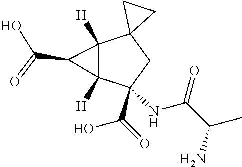 [SMILES: C[C@H](N)C(=O)N[C@@]1(C(=O)O)CC2(CC2)[C@H]2[C@H](C(=O)O)[C@H]21] | mGlu₂ and mGlu₃ Agonist | US20130197079A1 |
| LY404040 | (1R,2R,4S, 5S,6S)-4-amino- 2-hydroxy-214- thiabicyclo[3.1.0]hexane- 4,6-dicarboxylic acid | 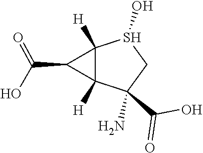 | mGlu₂ and mGlu₃ Agonist | Journal of Medicinal Chemistry (2007), 50(2), 233-240 |
| LY404039 (pomaglumetad) | (1R,4S,5S,6S)- 4-Amino-2- thiabicyclo [3.1.0]hexane- 4,6-dicaiboxylic acid 2,2- dioxide | 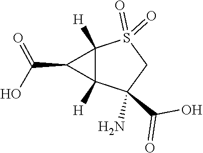 [SMILES: N[C@@]1(C(=O)O)CS(=O)(=O)[C@H]2[C@H](C(=O)O)[C@H]21] | mGlu₂ and mGlu₃ Agonist | WO2003104217A2 |
| LY2140023 (pomaglumetad methionil) | (1R,4S,5S, 6S)-4-[[(2S)-2- amino-4- methylsulfanylbutanoyl] amino]-2,2-dioxo-2λ⁶- thiabicyclo [3.1.0]hexane- 4,6-dicarboxylic acid | 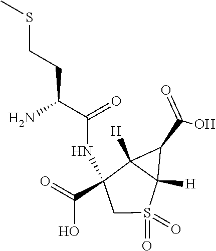 | mGlu₂ and mGlu₃ Agonist | WO2003104217A2 US20110237602A1 |
| LY341495 | (2S)-2-Amino- 2-[(1S,2S)-2- carboxycycloprop- 1-yl]-3- (xanth-9-yl) propanoic acid | 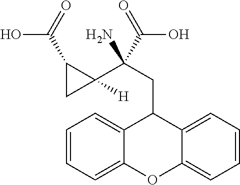 [SMILES: N[C@](CC1c2ccccc2Oc2ccccc21)(C(=O)O)[C@H]1C[C@@H]1C(=O)O] | mGlu₂ and mGlu₃ Antagonist | US5717109 |
| LY3020371 | (1S,2R,3S, 4S,5R,6R)-2- amino-3-[(3,4- difiluorophenyl) sulfanylmethyl]- 4-hydroxy- bicyclo[3.1.0] hexane-2,6- dicarboxylic acid | 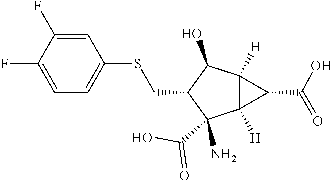 [SMILES: N[C@]1(C(=O)O)[C@@H]2[C@@H](C(=O)O)[C@@H]2[C@H](O)[C@H]1CSc1ccc(F)c(F)c1] | mGlu₂ and mGlu₃ Antagonist | WO2012068067A1 |
| HYDIA | (1S,2R,3R,5R, 6S)-2-amino- 3-hydroxy-bicyclo [3.1.0]hexane-2,6- dicarboxylic acid | 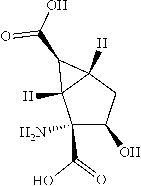 [SMILES: N[C@]1(C(=O)O)[C@H]2[C@@H](C[C@H]1O)[C@@H]2C(=O)O] | mGlu₂ and mGlu₃ Antagonist | DE19941675A1 |
| MGS0039 | (1R,2R,3R,5R, 6R)-2-amino- 3-[(3,4- dichlorophenyl) methoxy]-6 fluorobicyclo [3.1.0]hexane- 2,6-dicarboxylic acid | 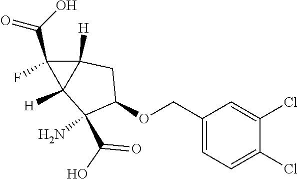 [SMILES: N[C@]1(C(=O)O)[C@H]2[C@@H](C[C@H]1OCc1ccc(Cl)c(Cl)c1)[C@]2(F)C(=O)O] | mGlu₂ and mGlu₃ Antagonist | WO2003061698A1 |
| LY341495 (CECXG) | (1S,2S,3S)-2- [1-Amino-1- carboxy-2- (9H-xantlien-9- yl)-ethyl]-3-ethyl- cyclopropanecarboxylic acid | 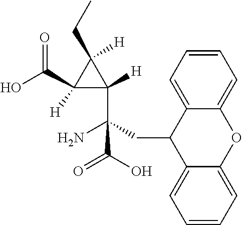 [SMILES: CC[C@@H]1[C@H](C(=O)O)[C@H]1[C@@](N)(CC1c2ccccc2Oc2ccccc21)C(=O)O] | mGlu₂ and mGlu₃ Antagonist | Bioorganic & Medicinal Chemistry Letters (1998), 8(20), 2849- 2854 |
| RO4491533 | 4-[3-(2,6- dimethylpylidin-4- yl)phenyl]-7-methyl-8- (trifluoromethyl)-1,3- dihydro-1,5- benzodiazepin- 2-one | 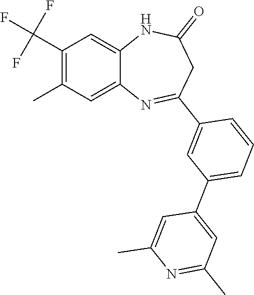 | mGlu₂ and mGlu₃ NAM | WO2003066623A1 |
| MINI-147 | 4-(8-Bromo- 2.3-dihydro-2- oxo-1H-1,5- benzodiazepin- 4-yl)-2- pyridinecarbonitrile | 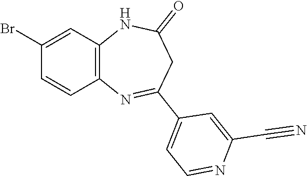 [SMILES: N#Cc1cc(C2=Nc3ccc(Br)cc3NC(=O)C2)ccn1] | mGlu₂ and mGlu₃ NAM | WO2015191630A1 |
| LY395756 | (1S,2S,4R,5R,6S)-rel- 2-Amino-4- methylbicyclo[3.1.0] hexane-2,6-dicarboxylic acid | 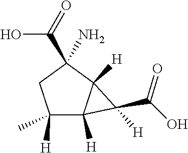 | mGlu₂ Agonist/ mGlu₃ Antagonist | EP774454A1 |

A group II metabotropic glutamate receptor modulator can be administered one or more times to a subject. A group II metabotropic glutamate receptor modulator can also be administered as part of a combination therapy for treating a disorder associated with a sleep spindle deficit. In some embodiments, a subject receiving a group II metabotropic glutamate receptor modulator can also be administered an additional therapeutic agent.

An additional therapeutic agent can be an antipsychotic. Examples of antipsychotics include, but are not limited to, aripiprazole, asenapine, brexpiprazole, cariprazine, clozapine, iloperidone, lurasidone, olanzapine, paliperidone, quetiapine, risperidone, ziprasidone, chlorpromazine, fluphenazine, haloperidol, and perphenazine.

An additional therapeutic agent can be a selective serotonin reuptake inhibitor (SSRI). Examples of SSRIs include, but are not limited to, citalopram, escitalopram, fluoxetine, fluvoxamine, fluvoxamine, paroxetine, paroxetine, sertraline.

An additional therapeutic agent can be a serotonin-norepinephrine reuptake inhibitor (SNRI). Examples of SNRIs include, but are not limited to, desvenlafaxine, duloxetine, venlafaxine, venlafaxine, milnacipran, and levomilnacipran.

An additional therapeutic agent can be a tricyclic antidepressant (TCA). Examples of TCAs include, but are not limited to, amitriptyline, desipramine, doxepine, imipramine, nortriptyline, amoxapine, clomipramine, maprotiline, trimipramine, and protriptyline.

An additional therapeutic agent can be a monoamine oxidase inhibitor (MAOI). Examples of MAOIs include, but are not limited to, phenelzine, selegiline, and tranylcypromine.

An additional therapeutic agent can be a benzodiazepine. Examples of benzodiazepines include, but are not limited to, alprazolam, clonazepam, diazepam, and lorazepam.

For intellectual disability (ID), intellectual and developmental disability (IDD) or mental retardation, an additional therapeutic agent can be metformin, memantine, flumazenil, or meclofenoxate. In some embodiments, an additional therapeutic agent for intellectual disability (ID), intellectual and developmental disability (IDD) or mental retardation can be in combination with Risperidone, Carbamazepine, Sodium Valproate, Lamotrigine, Lithium Carbonate, Methylphenidate, Procyclidine, Ferrous Fumarate+Vitamins+Lactulose+cod liver oil+various skin ointments, Clobazam Lorazepam, Rectal diazepam+buccal midazolam.

For schizophrenia disorders or schizotypal disorders, an additional therapeutic agent can be an antipsychotic including aripiprazole, asenapine, brexpiprazole, buspirone, cariprazine, chlorpromazine hydrochloride, clozapine, haloperidol, iloperidone, loxapine, lumateperone, lurasidone hydrochloride, molindone hydrochloride, olanzapine, paliperidone, perphenazine, prochlorperazine, quetiapine, risperidone, thiothixene, trifluoperazine, or ziprasidone.

For autism spectrum disorders, an additional therapeutic agent can be an antipsychotic including risperidone, aripiprazole, ziprasidone, SSRIs including fluoxetine, citalopram, escitalopram, stimulants including methylphenidate, or alpha-2-adrenergic agonists including clonidine, and guanfacine.

For bipolar disorders, an additional therapeutic agent can be an antipsychotic including aripiprazole, asenapine, catiprazine, loxapine, lurasidone hydrochloride, olanzapine, olanzapine and fluoxetine, quetiapine, risperidone, ziprasidone, clozapine, paliperidone, catiprazine, lurasidone, haloperidol, and chlorpromazine, an antidepressant including fluoxetine, an SSRI including citalopram, escitalopram, paroxetine, and sertraline, an SNRI including desvenlafaxine, duloxetine, and venlafaxine, a tricyclic including amitriptyline, desipramine, imipramine, and nortriptyline, an MAOI including phenelzine and tranylcypromine, an anticonvulsant including carbamazepine, divalproex sodium, lamotrigine, valproate sodium, valproic acid, and topiramate, a mood stabilizer including lithium and lithium carbonate, and/or a benzodiazepines including lorazepam, clonazepam, diazepam, alprazolam, and chlordiazepoxide.

For epilepsy, an additional therapeutic agent can be an anticonvulsant including carbamazepine, divalproex sodium, ethosuximide, felbamate, gabapentin, gabapentin and lidocaine, gabapentin and lidocaine and prilocaine, lacosamide, lamotrigine, levertiracetam, oxcarbazepine, perampanel, topiramate, valproate, valproic acid, zonisamide, cannabidiol, cenobamate, phenytoin, ezogabine, rufinamide, stiripentol, vigabatrin, eslicarbazepine acetate, pregabalin, and tiagabine, a benzodiazepine including diazepam, midazolam, clobazam, lorazepam, and/or a barbiturate including phenobarbital and primidone.

A group II metabotropic glutamate receptor modulator can be administered before or after the administration of the additional therapeutic agent. In some embodiments, the group II metabotropic glutamate receptor modulator and the additional therapeutic agent are administered concurrently, or in close temporal proximity (e.g., there may be a short time interval between the administrations, such as during the same treatment session). In some embodiments, there may be greater time intervals between the administrations, such as during the same or different treatment sessions.

### Subjects

A subject to be treated by methods described herein may be a human subject or a non-human subject. Non-human subjects include, for example: non-human primates; farm animals, such as cows, horses, goats, sheep, and pigs; pets, such as dogs and cats; and rodents.

A subject to be treated by methods described herein may be a human subject having, suspected of having, or at risk for developing a disorder associated with a sleep spindle deficit. In some embodiments, a subject has been diagnosed as having a disorder associated with a sleep spindle deficit, while in other embodiments, a subject has not been diagnosed as having a disorder associated with a sleep spindle deficit. In some embodiments, the subject is a human subject having, suspected of having, or at risk for developing a schizophrenia disorder. In some embodiments, the subject is a human subject having, suspected of having, or at risk for developing epilepsy. In some embodiments, the subject is a human subject having, suspected of having, or at risk for developing an autism spectrum disorder. In some embodiments, the subject is a human subject having, suspected of having, or at risk for developing a bipolar disorder. In some embodiments, the subject is a human subject having, suspected of having, or at risk for developing an intellectual disability. In some embodiments, the subject is a human subject having, suspected of having, or at risk for developing a CACNA1I gene disorder. In some embodiments, the subject is a human subject having a mutation in the gene encoding the CACNA1I protein.

In some embodiments, the subject is a subject exhibiting at least one defective characteristics of sleep spindle oscillations and/or at least one symptom associated with defective sleep spindle oscillations. For example, the subject may exhibit memory and/or learning impairment, difficulty sleeping (including but not limited to lack of sleep quality, difference in sleep architecture and stage transitions), or a combination thereof. In other embodiments, the subject has not exhibited any symptoms of a disorder associated with a sleep spindle deficit and/or has no history of a disorder associated with a sleep spindle deficit.

In some embodiments, the subject is a human subject exhibiting at least one symptom of a schizophrenia disorder. For example, the subject may exhibit fatigue, paranoia, depression, anxiety, and/or memory loss, or a combination thereof. In some embodiments, the subject has not exhibited any symptoms of schizophrenia and/or has no history of a schizophrenia disorder.

### Identification of Subjects with a Disorder Associated with a Sleep Spindle Deficit

Aspects of the disclosure relate to treating subjects having, suspected of having, or at risk of developing a disorder associated with a sleep spindle deficit. In some embodiments, a subject in need of treatment is a subject having, suspected of having, or at risk of developing a disorder associated with a sleep spindle deficit. As used herein, treating a disorder, such as a disorder associated with a sleep spindle deficit, refers to ameliorating or improving at least one symptom of the disorder, such as a disorder associated with a sleep spindle deficit. Treating a disorder, such as a disorder associated with a sleep spindle deficit, includes the application or administration of one or more therapeutic agents to a subject who has a disorder associated with a sleep spindle deficit. In some embodiments, the subject may not yet exhibit, or has not been diagnosed with a disorder associated with a sleep spindle deficit. As used herein, an effective amount of an agent, such as a modulator, refers to an amount of the agent that is effective to ameliorate or improve at least one symptom of a disorder in a subject. In some embodiments, an effective amount of a group II metabotropic glutamate receptor modulator as used herein refers to the amount of a group II metabotropic glutamate receptor modulator that can confer a beneficial and/or therapeutic effect on a subject, either alone or in combination with one or more other active agents. In some aspects of the present disclosure, “an effective amount” of a group II metabotropic glutamate receptor modulator as used herein refers to the amount of a group II metabotropic glutamate receptor modulator that is effective to ameliorate or improve at least one symptom of a disorder associated with sleep spindle deficit in a subject.

Effective amounts may vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed without undue experimentation. In some embodiments, it is preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for any other reasons.

Characteristics of sleep spindles in a subject may be evaluated to determine whether the subject is suitable for treatment with a group II metabotropic glutamate receptor modulator. In some embodiments, subjects may be identified as having a disorder associated with a sleep spindle deficit by comparing one or more characteristics of a sleep spindle from the subject to the same characteristic of a sleep spindle from a control subject or by comparing one or more characteristics of a sleep spindle from the subject to a predetermined reference for such a sleep spindle characteristic.

Accordingly, in some embodiments, a subject having a disorder associated with a sleep spindle deficit is a subject in which a characteristic of a sleep spindle is altered compared to the same characteristic in a control subject (e.g., a subject who does not have an altered sleep spindle) or compared to a predetermined reference for such a characteristic. Characteristics of sleep spindles include, but are not limited to, sleep spindle activity, sleep spindle density, sleep spindle duration, sleep spindle amplitude, sleep spindle frequency, number of oscillations, and spindle symmetry.

In some embodiments, if a characteristic of a sleep spindle in a subject deviates (e.g., is increased or decreased) compared to the same characteristic in a control subject, the subject may be identified as suitable for treatment with a group II metabotropic glutamate receptor modulator. In some embodiments, the control subject is a healthy individual, e.g., an individual that is apparently free of a disorder associated with a sleep spindle deficit or an individual that has no history of a disorder associated with a sleep spindle deficit (e.g., schizophrenia). A control subject may be, in some embodiments, a population of healthy subjects or an average calculated from a population of healthy subjects. In other embodiments, a control subject is a subject who has or has had in the past a disorder associated with a sleep spindle deficit.

In some embodiments, if a characteristic of a sleep spindle deviates (e.g., is increased or decreased) compared to a predetermined reference for that characteristic, the subject may be identified as suitable for treatment with a group II metabotropic glutamate receptor modulator. A predetermined reference for a characteristic can be a specific value or range of values that indicates whether that characteristic is normal or abnormal. For example, if the characteristic being measured in the subject is sleep spindle activity, then a predetermined reference could be a specific activity level or range of activity levels that would be considered normal or healthy for that characteristic. In some embodiments, an abnormal or unhealthy value or level, or an abnormal or unhealthy range of values or levels, may be indicative of a disorder associated with a sleep spindle deficit.

Methods described herein encompass detecting characteristics of sleep spindles to determine whether a subject has a disorder associated with a sleep spindle deficit. For example, methods can comprise: detecting sleep spindle density; detecting sleep spindle duration; detecting sleep spindle amplitude; detecting sleep spindle frequency; detecting the number of oscillations of a sleep spindle; and/or detecting spindle symmetry.

Any number of sleep spindle characteristics may be detected in methods described herein. In some embodiments, methods comprise detecting at least 1, at least 2, at least 3, at least 4, at least 5, at least 6, at least 7, at least 8, at least 9, at least 10, or more than 10 characteristics of sleep spindles.

Methods described herein encompass detecting characteristics of sleep spindles by any technique or method known in the art. In some embodiments, detecting a characteristic of a sleep spindle comprises detection via electroencephalogram (EEG) or via electrocardiogram (ECG).

Methods described herein can also be applied for evaluation of the efficacy of a group II metabotropic glutamate receptor modulator for treatment of a disorder associated with a sleep spindle deficit. For example, characteristics of sleep spindles may be detected in a subject to whom a treatment is administered, before and after the treatment and/or during the course of the treatment. For example, in some embodiments, if a characteristic of sleep spindles improves during the course of treatment, then this may be an indication that the treatment is effective and should be continued.

In some instances, if a subject is identified as not responsive to a treatment, a higher dose and/or frequency of dosage of a group II metabotropic glutamate receptor modulator can be administered to the subject. In some embodiments, the dosage or frequency of dosage of the group II metabotropic glutamate receptor modulator is maintained, lowered, increased, or ceased in a subject. Alternatively, a different or supplemental treatment can be applied to a subject who is found not to be responsive to a group if metabotropic glutamate receptor modulator.

Also within the scope of the present disclosure are methods of evaluating the severity of a disorder associated with a sleep spindle deficit. For example, a disorder associated with a sleep spindle deficit may be in a quiescent state (remission), during which the subject may not experience symptoms of the disease. Relapses are typically recurrent episodes in which the subject may experience a symptom of a disorder associated with a sleep spindle deficit. In some embodiments, detection of sleep spindle characteristics can be indicative of whether the subject will experience, is experiencing, or will soon experience a relapse of a disorder associated with a sleep spindle deficit. In some embodiments, methods involve comparing characteristics of sleep spindles in a subject having a disorder associated with a sleep spindle deficit to sleep spindles from the same subject at a different stage or time point when the subject does not exhibit symptoms of a disorder associated with a sleep spindle deficit.

### CACNA1I

Sleep spindle generation is supported by CaV3.3 voltage-gated calcium channels encoded by the CACNA1I gene. Cav3.3 channels are expressed in a limited subset of neurons including GABAgeric neurons of the TRN where they support oscillatory activity essential for sleep spindle generation. The amino acid sequence of human CACNA1I protein is provided, for example, in UniProt Q9P0X4-1, RefSeq NP_066919.2, and SEQ ID NO: 1. CACNA1I is implicated in schizophrenia risk. Mutations in the CACNA1 I gene that disrupt CaV3.3 channel activity are associated with increased risk of developing a disorder associated with a deficit in sleep spindles (e.g., schizophrenia). Some aspects of the disclosure relate to identifying subjects having a disorder associated with a sleep spindle deficit, such as schizophrenia disorders, based at least in part on whether the subject has a mutation in the gene encoding the CACNA1I protein.

In some embodiments, the mutation in the gene encoding the CACNA1I protein comprises a mutation at the amino acid residue corresponding to amino acid residue 1346 in the CACNA1I protein depicted by SEQ ID NO: 1. For example, in certain embodiments, the mutation at the amino acid residue corresponding to amino acid residue 1346 in the CACNA1I protein depicted by SEQ ID NO: 1 is a substitution of arginine (R) to histidine (H) (i.e., R1346H).

As used herein, a residue (such as a nucleic acid residue or an amino acid residue) in sequence “X” is referred to as corresponding to a position or residue (such as a nucleic acid residue or an amino acid residue) “a” in a different sequence “Y” when the residue in sequence “X” is at the counterpart position of “a” in sequence “Y” when sequences X and Y are aligned using amino acid sequence alignment tools known in the art, such as, for example, Clustal Omega or BLAST®.

It should be appreciated that detection of a mutation in the gene encoding the CACNA1I protein can be achieved by any means known in the art. For example, a mutation in the DNA of the gene encoding the CACNA1I protein could be detected by DNA sequencing of the gene and comparing the sequence to a control wild type sequence for the CACNA1I gene. A mutation in the CACNA1I protein could also he detected through other standard methods, such as mass spectrometry.

In some embodiments, the human CACNA1I protein comprises or consists of the amino acid sequence of SEQ ID NO: 1. In some embodiments, the mutation in human CACNA1I protein comprises a mutation at amino acid position 1346 in SEQ ID NO: 1. In some embodiments, the mutation at amino acid position 1346 of SEQ ID NO: 1 is a substitution of arginine (R) to histidine (H) (i.e., R1346H) in SEQ ID NO: 1.

### General Techniques

The practice of the present disclosure will employ, unless otherwise indicated, conventional techniques of molecular biology (including recombinant techniques), microbiology, cell biology, biochemistry and immunology, which are within the ordinary skill in the art (e.g., as disclosed in: Molecular Cloning: A Laboratory Manual, fourth edition (Green, et al., 2012 Cold Spring Harbor Press); Oligonucleotide Synthesis (M. J. Gait, ed., 1984); Methods in Molecular Biology, Humana Press; Cell Biology: A Laboratory Notebook, Vol. 3 (J. E. Cellis, ed., 2005) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Short Protocols in Molecular Biology (F. M. Ausubel, et al., eds., 2002); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995). It is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described herein may be more fully understood, the following examples are set forth. The examples described in this application are offered to illustrate the systems and methods provided herein and are not to be construed in any way as limiting their scope.

### Example 1: Group II Metabotropic Glutamate Receptor Agonists Increase Rebound Burst Firing in Thalamic Reticular Nucleus (TRN) Neurons

TRN neurons are known to display dual firing modes depending on their resting membrane potential: tonic and burst firing. While tonic firing refers to regular sodium spike trains at depolarized membrane potential, burst firing is characterized by repetitive “low-threshold” T type Ca²⁺ transients (mediated by Cav3.3 channel) crowned by high-frequency sodium spikes when hyperpolarized. This burst firing mode may be critical for the generation and/or the maintenance of certain sleep rhythms in mice, including generation and maintenance of sleep spindle oscillations (Astori et al., 2011; Lee et al., 2014; Steriade et al., 1993; von Krosigk et al., 1993; Sherman et al., 2002).

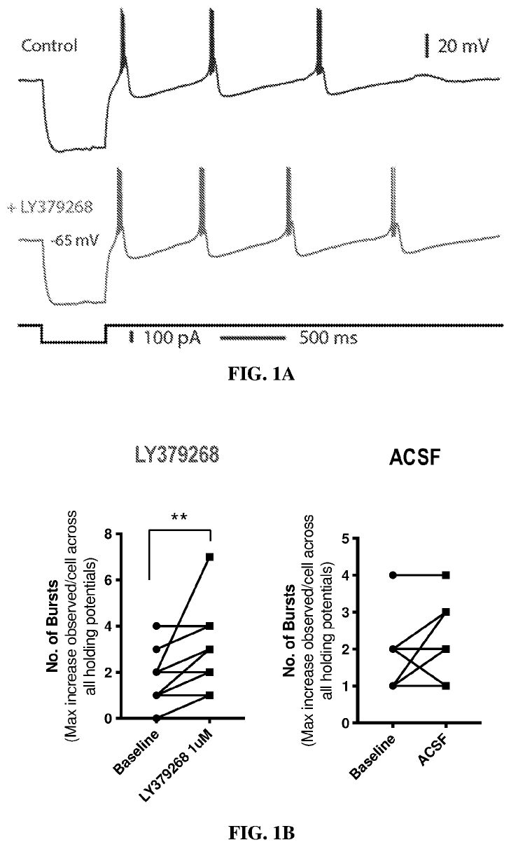

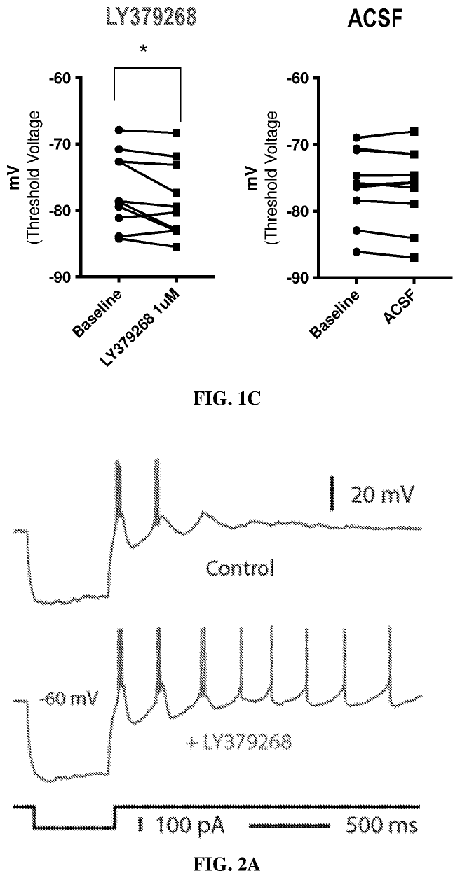

Whether this characteristic TRN burst firing activity is affected by activation of the group II metabotropic glutamate receptors, including mGlu₂ and mGlu₃, was examined using the group II metabotropic glutamate receptor agonist, LY379268. As shown in FIG. 1A-1B, a significant increase in the repetitive rebound bursting after the addition of LY379268 compared to baseline levels in acute brain slices acquired from wild-type (WT) mice was observed. The rebound bursting in the baseline condition (control) was 1.87±0.291 (mean±s.e.m.), while after the bath application of LY379268 (1 μM) it was 2.87±0.435 (n=15 Wilcoxon signed rank test: p=0.002**). Such increase was not observed in WT slices treated with artificial cerebrospinal fluid (ACSF; n=14; baseline: 1.79±0.291; ACSF treated: 2.21±0.299; Wilcoxon signed rank test: p=0.1562). In addition, there was also a reduction in burst threshold (defined as the lowest membrane holding potential from which hyperpolarization induced rebound bursting can be observed) in WT mice after bath application of LY379268 (1 μM; FIG. 1C). The threshold for rebound bursting in the baseline condition (control) was −76.98 mV±1.79 (mean±s.e.m.), while after the bath application of LY379268 (1 μM) it was −78.48 mV±1.80 (n=10 Wilcoxon signed rank test: p=0.048*). Such changes in threshold were also not observed following ACSF treatment (n=10; baseline: −76.06 mV±1.71; ACSF treated: −76.32 mV±1.82; Wilcoxon signed rank test: p=0.3750).

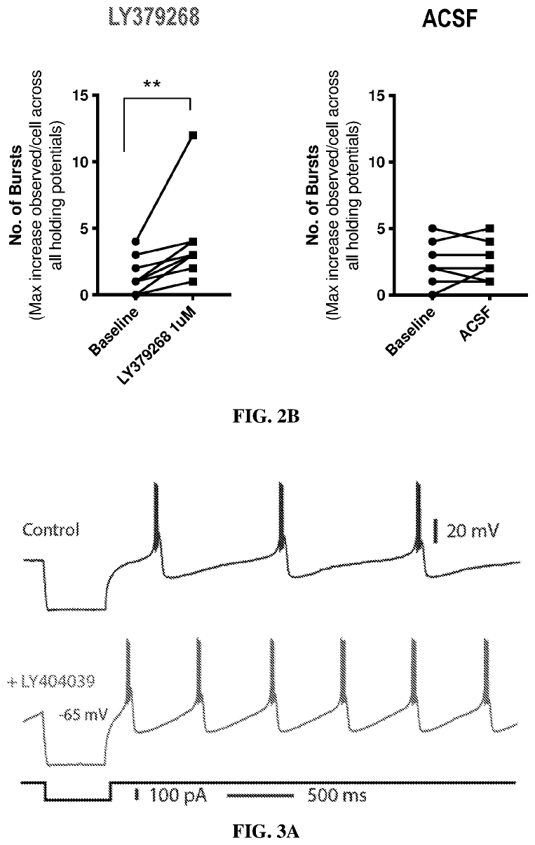

Mice carrying a mutation in CACNA1I (R1305H; RH mice) show a significant deficit in rebound bursting of TRN neurons. The amino acid sequence of mouse CACNA1I protein is provided in SEQ ID NO: 2. Whether the group II metabotropic glutamate receptor agonist LY379268 could rescue such deficits in rebound bursting in RH mice was tested. Similar to in WT mice, LY379268 (1 μM) was also able to increase rebound bursting in RH mice (FIG. 2A-2B). The rebound bursting in baseline/control conditions was 1.44±0.444 (mean±s.e.m.), while after the bath application of LY379268 (1 μM) it was 3.78±1.08 (n=9 Wilcoxon signed rank test: p=0.0039**). Such increase was not observed in RH slices treated with artificial cerebrospinal fluid (ACSF; n=10; baseline: 2.50±0.477; ACSF treated: 2.70±0.473; Wilcoxon signed rank test: p=0.75).

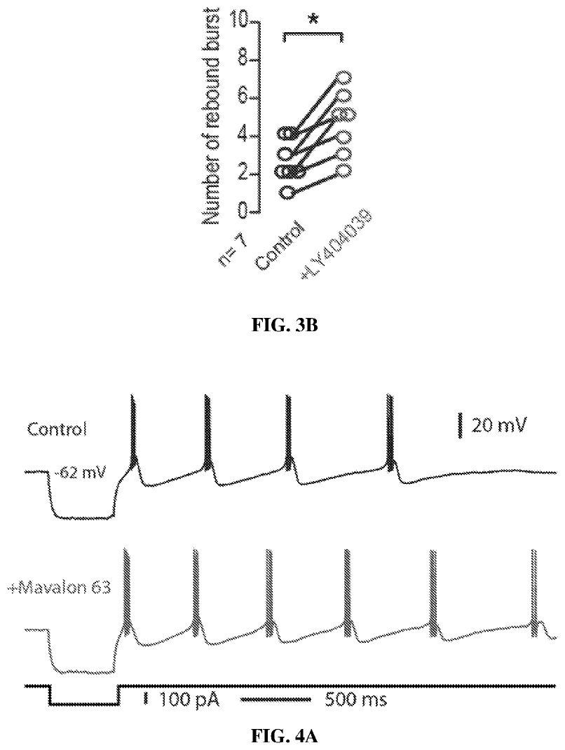

Since early clinical trials used a prodrug form of another selective group II metabotropic glutamate receptor agonist, specifically LY-404,039 (a methionine amide of LY-404039, also called pornagiumetad methionil or LY-2140023 monohydrate), the effect of LY-404039 on TRN neuron rebound bursting activity in WT mice was measured. Consistent with the effect of LY379268, the application of LY-404039 significantly enhanced the number of rebound bursts in WT mice (FIG. 3A-3B). The rebound bursting in control conditions was 2.71±0.42 (mean±s.e.m.), while after the bath application of LY404039 (1 μM) it was 4.57±0.64 (n=7 Wilcoxon signed rank test: p=0.0156).

Taken together, these results demonstrate that group II metabotropic glutamate receptor agonists increased rebound burst firing in TRN neurons in WT and RH mice.

### Example 2: mGlu₃ Positive Allosteric Modulator (PAM) Mavalon-63 Increases Rebound Bursting in TRN Neurons

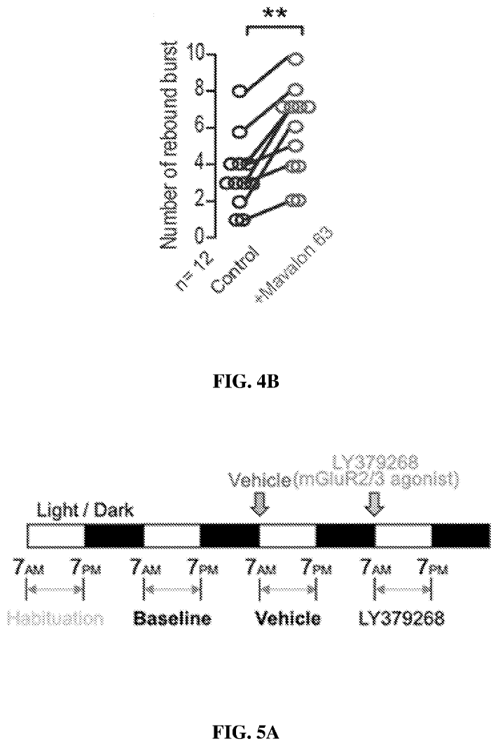

Whether specific allosteric activation of the mGlu₃ receptor can also enhance rebound bursting activity in TRN neurons was examined. The number of rebound bursting was significantly enhanced in WT TRN neurons using a positive allosteric modulator (PAM) Mavalon 63 (FIG. 4A-4B). The rebound bursting in baseline/control conditions was 3.2±0.77 (mean±s.e.m.), while after the bath application of Mavalon 63 (500 nM) it was 4.9±0.91 (n=12 Wilcoxon signed rank test: p=0.0020). These results demonstrate that specific activation of mGlu₃ alone is sufficient to increase rebound bursting.

### Example 3: Group II Metabotropic Glutamate Receptor Agonist LY379268 Increases Sleep Spindles in WT and RH Mice

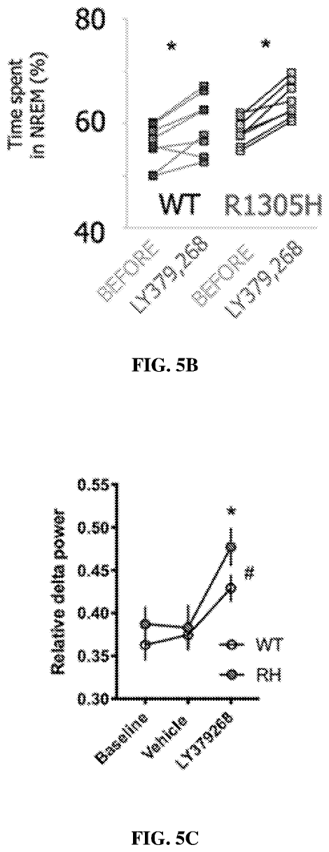

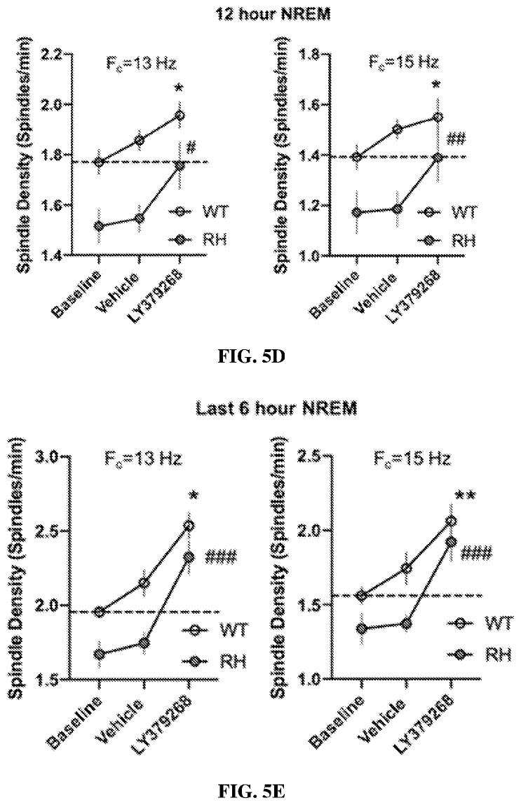

There is a strong correlation between rebound bursting in TRN neurons and the rate of occurrence of sleep spindle oscillations during NREM or deep sleep state in mice (Wells et al., Ghoshal et al.) Since LY379268 increased rebound burst firing of TRN neurons in WT mice and rescued rebound bursting deficits in RH mice, it was examined whether activation of mGlu₃ could rescue the sleep spindle deficits observed in the RH mice. LY379268 was administered intraperitoneally (i.p.; 10 mg/kg) while simultaneously recording the brain activity using electroencephalography (EEG) in WT and Ca_{V}3.3 RH mice. EEG was recorded using surface electrodes chronically implanted near the frontal and parietal lobes of the mice (Pinnacle Systems). Mice were habituated in the EEG recording arena for 48 hrs and then injected with vehicle (0.9% saline) and recorded for another 24 hours. Then, 10 mg/kg LY379268 formulated in 0.9% saline was injected, and their EEG activity was recorded for another 24 hours, and analyzed by Luna (zzz.bwh.harvard.edu, Ghoshal et al., 2020). The experimental design is shown in FIG. 5A.

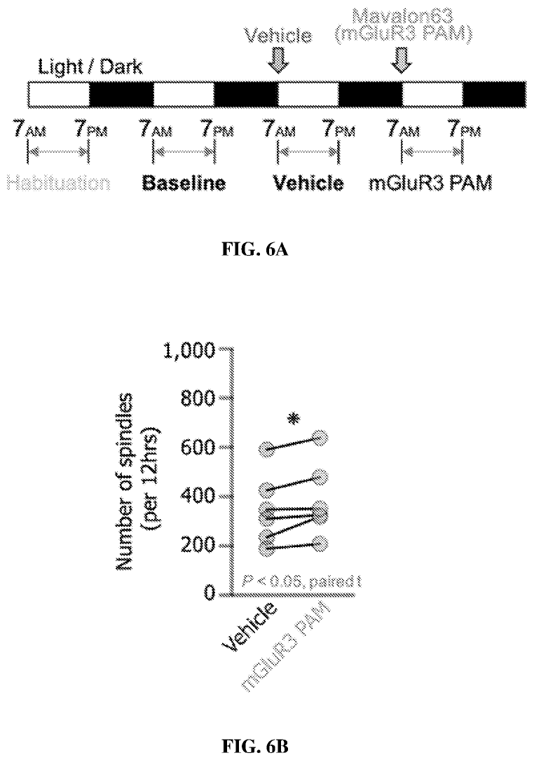

Administration of 10 mg/kg LY379268 (i.p.) increased the time spent in NREM sleep in both WT and RH mice during the 12 hr light cycle and a concurrent increase in relative delta power (FIG. 5B-5C). For the entire NREM period during 12-hr light cycle, a modest but significant increase in sleep spindle density in both WT and RH mice was observed, especially in the parietal cortex (F_{c}=13 Hz (Fixed effect of treatment p<0.0001) and 15 Hz (Fixed Effect of treatment p<0.0001); Mixed effects model; FIG. 5D). There were no significant effects observed during the vehicle condition (FIG. 5D). Administration of LY379268 also rescued. NREM specific sleep spindle deficits in RH mice to wildtype levels (FIG. 5D; WT-baseline vs RH-baseline: p<0.001; WT-baseline vs RH-LY379268: p>0.05). The increase in sleep spindles following the administration of LY379268 was more robust during the latter half (last 6 hours) of the light cycle (FIG. 6E; F_{c}=13 Hz (Fixed effect of treatment p<0.0001) and 15 Hz (Fixed Effect of treatment p<0.0001).

Taken together, these results demonstrate that the group II tnetabotropic glutamate receptor agonist LY379268 increased sleep spindles in WT and RH mice.

### Example 4: mGlu₃ Positive Allosteric Modulator (PAM) Mavalon-63 Increases Sleep Spindles in WT Mice

Whether allosteric activation of mGlu₃ receptor using the Mavalon-63 PAM would also lead to an increase in sleep spindle density was examined. The experimental design is shown in FIG. 6A, and mice were similarly recorded as in Example 4. Raw EEG recording traces were bandpass filtered, and analyzed using a decision tree algorithm including short-time Fourier transform, root-mean-square amplitude and Hilbert transform. Unlike the effects of LY379268, 10 mg/kg Mavalon-63 did not increase the time spent in NREM for mice during the 12-hr light cycle, but there was an appreciable and significant (p<0.05; paired t-test) increase in parietal sleep spindle density (averaged across all center frequencies 9-15 Hz) during NREM (FIG. 6B). These results show that selective activation of mGlu₃ is sufficient to increase sleep spindle density in mice.

### REFERENCES

Astori, S, Wimmer R D, Prosser H M, Corti C, Corsi M, Liaudet N, Volterra A, Franken P, Adelman J P, Lüthi A. (2011). The Ca(V)3.3 calcium channel is the major sleep spindle pacemaker in thalamus. Proc. Natl. Acad. Sci. 108(33):13823-8.
Bond A, Ragumoorthy N, Monn J A, Hicks C A, Ward M A, Lodge D, O'Neill M J. (1999). LY379268, a potent and selective Group II metabotropic glutamate receptor agonist, is neuroprotective in gerbil global, but not focal, cerebral ischaemia. Neurosci. Lett. 8;273(3).191-4.
Lee S E, Lee J, Latchoumane C, Lee B, Oh S J, Saud Z A, Park C, Sun N, Cheong E, Chen C C, Choi E J, Lee C J, Shin H S. (2014). Rebound burst firing in the reticular thalamus is not essential for pharmacological absence seizures in mice. Proc. Natl. Acad. Sci. 12;111(32):11828-33.
Uygun D S, McNally J M, Yang L, Imaizumi K, Katsuki F, Brown R E, Mao X, Nicholson T, Sidor M, Zhang Q, Strecker R E, McCarley R W, Feng G, Pan J Q. (2017). Abnormal Sleep Spindle Rhythmogenesis in Mice Bearing a Schizophrenia Associated Coding Variant in the CACNA1I Gene. Sleep. 40(1):A6-A7.
Wells M F, Wimmer R D, Schmitt L I, Feng G, Halassa M M. (2016). Thalamic reticular impairment underlies attention deficit in Ptchd1(Y/-) mice. Nature. 532(7597):58-63.
Steriade, M., McCormick, D. A. & Sejnowski, T. J. Thalamocortical oscillations in the sleeping and aroused brain. Science 262, 679-685 (1993).
von Krosigk, M., Bal, T. & McCormick, D. A. Cellular mechanisms of a synchronized oscillation in the thalamus. Science 261, 361-364 (1993).
Sherman, S. M. & Guillery, R. W. The role of the thalamus in the flow of information to the cortex. Philosophical transactions of the Royal Society of London. Series B, Biological sciences 357, 1695-1708, doi:10.1098/rstb.2002.1161 (2002).
Kulkarni et al., A deep learning approach for real-time detection of sleep spindles, J Neural Eng. 2019 June; 16(3): 036004. Doi: 10.1088/1741-2552/ab0933.

SEQUENCES
Human CACNA1I Protein
Uniprot: Q9P0X4-1
RefSeq: NP_066919.2
(SEQ ID NO: 1)
MAESASPPSSSAAAPAAEPGVTTEQPGPRSPPSSPPGLEEPLDGADPHVPHPDLAPIAFF
CLRQTTSPRNWCIKMVCNPWFECVSMLVILLNCVTLGMYQPCDDMDCLSDRCKILQVFDD
FIFIFFAMEMVLKMVALGIFGKKCYLGDTWNRLDFFIVMAGMVEYSLDLQNINLSAIRTV
RVLRPLKAINRVPSMRILVNLLLDTLPMLGNVLLLCFFVFFIFGIIGVQLWAGLLRNRCF
LEENFTIQGDVALPPYYQPEEDDEMPFICSLSGDNGIMGCKEIPPLKEQGRECCLSKDDV
YDFGAGRQDLNASGLCVNWNRYYNVCRTGSANPHKGAINFDNIGYAWIVIFQVITLEGWV
EIMYYVMDAHSFYNFIYFILLIIVGSFFMINLCLVVIATQFSETKQREHRLMLEQRQRYL
SSSTVASYAEPGDCYEEIFQYVCHILRKAKRRALGLYQALQSRRQALGPEAPAPAKPGPH
AKEPRHYHGKTKGQGDEGRHLGSRHCQTLHGPASPGNDHSGRELCPQHSPLDATPHTLVQ
PIPATLASDPASCPCCQHEDGRRPSGLGSTDSGQEGSGSGSSAGGEDEADGDGARSSEDG
ASSELGKEEEEEEQADGAVWLCGDVWRETRAKLRGIVDSKYFNRGIMMAILVNTVSMGIE
HHEQPEELTNILEICNVVFTSMFALEMILKLAAFGLFDYLRNPYNIFDSIIVIISIWEIV
GQADGGLSVLRTFRLLRVLKLVRFMPALRRQLVVLMKTMDNVATFCMLLMLFIFIFSILG
MHIFGCKFSLRTDTGDTVPDRKNFDSLLWAIVTVFQILTQEDWNVVLYNGMASTSPWASL
YFvALMTFGNYVLFNLLVAILVEGFQAEGDANRSYSDEDQSSSNIEEFDKLQEGLDSSGD
PKLCPIPMTPNGHLDPSLPLGGHLGPAGAAGPAPRLSLQPDPMLVALGSRKSSVMSLGRM
SYDQRSLSSSRSSYYGPWGRSAAWASRRSSWNSLKHKPPSAEHESLLSAERGGGARVCEV
AADEGPPRAAPLHTPHAHHIHHGPHLAHRHRHHRRTLSLDNRDSVDLAELVPAVGAHPRA
AWRAAGPAPGHEDCNGRMPSIAKDVFTKMGDRGDRGEDEEEIDYTLCFRVRKMIDVYKPD
WCEVREDWSVYLFSPENRFRVLCQTIIAHKLFDYVVLAFIFLNCITIALERPQIEAGSTE
RIFLTVSNYIFTAIFVGEMTLKVVSLGLYFGEQAYLRSSWNVLDGFLVFVSIIDIVVSLA
SAGGAKILGVLRVLRLLRTLRPLRVISRAPGLKLVVETLISSLKPIGNIVLICCAFFIIF
GILGVQLFKGKFYHCLGVDTRNITNRSDCMAANYRWVHHKYNFDNLGQALMSLFVLASKD
GWVNIMYNGLDAVAVDQQPVTNHNPWMLLYFISFLLIVSFFVLNMFVGVVVENFHKCRQH
QEAEEARRREEKRLRRLEKKRRKAQRLPYYATYCHTRLLIHSMCTSHYLDIFITFIICLN
VVTMSLEHYNQPTSLETALKYCNYMFTTVFVLEAVLKLVAFGLRRFFKDRWNQLDLAIVL
LSVMGITLEEIEINAALPINPTIIRIMRVLRIARVLKLLKMATGMRALLDTVVQALPQVG
NLGLLFMLLFFIYAALGVELFGKLVCNDENPCEGMSRHATFENFGMAFLTLFQVSTGDNW
NGIMKDTLRDCTHDERSCLSSLQFVSPLYFVSFVLTAQFVLINVVVAVLMKHLDDSNKEA
QEDAEMDAELELEMAHGLGPGPRLPTGSPGAPGRGPGGAGGGGDTEGGLCRRCYSPAQEN
LWLDSVSLIIKDSLEGELTIIDNLSGSIFHHYSSPAGCKKCHHDKQEVQLAETEAFSLNS
DRSSSILLGDDLSLEDPTACPPGRKDSKGELDPPEPMRVGDLGECFFPLSSTAVSPDPEN
FLCEMEEIPFNPVRSWLKHDSSQAPPSPFSPDASSPLLPMPAEFFHPAVSASQKGPEKGT
GTGTLPKIALQGSWASLRSPRVNCTLLRQATGSDTSLDASPSSSAGSLQTTLEDSLTLSD
SPRRALGPPAPAPGPRAGLSPAARRRLSLRGRGLFSLRGLRAHQRSHSSGGSTSPGCTHH
DSMDPSDEEGRGGAGGGGAGSEHSETLSSLSLTSLFCPPPPPPAPGLTPARKFSSTSSLA
APGRPHAAALAHGLARSPSWAADRSKDPPGRAPLPMGLGPLAPPPQPLPGELEPGDAASK
RKR
Mouse CACNAII Protein
Uniprot: E9Q7P2
RefSeq: NP_001037773.2
(SEQ ID NO: 2)
MADSNLPPSSSAAPDPEPGITEQPGPRSPPPSPPGLEEPLDGTNPDVPHPDLAPVAFFCL
RQTTSPRKWCIKMVCNPWFECVSMLVILLNCVTLGMYQPCDDMECLSDRCKILQVFDDFI
FIFFAMEMVLKMVALGIFGKKCYLGDTWNRLDFFIVMAGMVEYSLDLQNINLSAIRTVRV
LRPLKAINRVPSMRILVNLLLDTLPMLGNVLLLCFFVFFIFGIIGVQLWAGLLRNRCFLE
ENFTIQGDVALPPYYQPEEDDEMPFICSLSGDNGIMGCHEIPPLKEQGRECCLSKDDMYD
FGAGRQDLNASGLCVNWNRYYNVCRTGNANPHKGAINFDNIGYAWIVIFQVITLEGWVEI
MYYVMDAHSFYNFIYFILLIIVGSFFMINLCLVVIATQFSETKQREHRLMLEQRQRYLSS
STVASYAEPGDCYEEIFQYVCHILRKAKRRALGLYQALQNRRQATGPGTPAPAKPGPHAK
EPSHCKLCPRHSPLDTTPHTLVQPISAILASDPSSCPRCQHEAGRRPSGLGSTDSGQEGS
GSGGSAEAEANGDGPQSSEDGVSSGLGKEEEQEDGAARLCGDVWRETRAKLRGIVDSKYF
NRGIMMAILVNTVSMGIEHHEQPEELTNILEICIPVFTSMFALEMILKLAAFGLFDYLRN
PYNIFDSIIVIISIWEIVGQADGGLSVLRTFRLLRVLKLVRFMPALRRQLVVLMKTMDNV
ATFCMLLMLFIFIFSILGMHIFGCKFSLRTDTGDTVPDRKNFDSLLNAIVTVFQILTQED
WNVVLYNGMASTTPWASLYFVALMTFGNYVLFNLLVAILVEGFQAEGDANRSYSDEDQSS
SNLEELDKLPEGLDSSRDLKLCPIPMTPNGHLDPSLPLGGHLGPAGAMGAAPRLSLQPDP
VLVALESRKSSVMSLGRMSYDQRSLSSSRSSYYGPWGRSGTWASRRSSNNSLKHKPPSAE
HESLLSGERGGSCVRACEGAREDAPPRAAPLHAPHTHHAHHGPHLAHRHRHHRRTLSLDT
RDSVDLAELVPVVGAHSRAAWRAAGQAPGHEDCNGRMPNIAKDVFTKMDDRRDRGEDEEE
IDYTLCFRVRKMIDVYKPDWCEVREDWSVYLFSPENKFRILCQTIIAHKLFDYVVLAFIF
LNCITIALERPQIEAGSTERIFLTVSNYIFTAIFVGEMTLKVVSLGLYFGEQAYLRSSWN
VLDGFLVFVSIIDIVVSVASAGGAKILGVLRVLRLLRTLRPLRVISRAPGLKLVVETLIS
SLKPIGNIVLICCAFFIIFGILGVQLFKGKFYHCLGVDTRNITNRSDCVAANYRWVHHKY
NFDNLGQALMSLFVLASKDGWVNIMYNGLDAVAVDQQPVTNHNPWMLLYFISFLLIVSFF
VLNMFVGVVVENFHKCRQHQEAEEARRREEKRLRRLEKKRRKAQRLPYYATYCPTRLLIH
SMCTSHYLDIFITFIICLNVVTMSLEHYNQPTSLETALKYCNYMFTTVFVLEAVLKLVAF
GLRRFFKDRWNQLDLAIVLLSVMGITLEEIEINAALPINPTIIRIMRVLRIARVLKLLKM
ATGMRALLDTVVQALPQVGNLGLLFMLLFFIYAALGVELFGKLVCNDENPCEGMSRHATF
ENFGMAFLTLFQVSTGDNKNGIMKDTLRDCTHDERSCLSSLQFVSPLYFVSFVLTAQFVL
INVVVAVLMKHLDDSNKEAQEDAEMDAEIELEMAHGLGPGPGPCPCPCPCPCPCPCPGPR
MPTSSPGAPGRGSGGAGVGGDTESHLCRHCYSPAQETLWLDSVSLIIKDSLEGELTIIDN
LSGSIFHHYSSPAGCDKCHHDKQEVQLAETEAFSLNSDRSSSVLLGDDLSLEDPTACPQG
PKESKGELEPPEPMQAGDLDECFFPFAGEPVSAGPESLLCEMGAIPFNPVQSWLKHESNQ
APPSPFSPDGSSPLLQMPAEFFHPAVSASQKGQEPGMSSGTLPKIALQGSWASLRSPSVN
CTLLRQATVSDTSLDASPSSSAGSLQTTLEDSLTLSDSPRRALGPPVQVPGPRASLSPAT
RRRLSLRGRGLFSLRGLRAHQRSHSSGGSTSPGCTYHDSMDPSDEEGRGGAGGGGAGSEH
SETLSSLSLTSLFCLPPTLPPPGLTPARKFSSTSSLAAGPGRPGATVSVRGLARSPSWAA
DRSKDPPGQAQLASGFGSSAPEPQPPPGESTDAASKRKR

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

### Equivalents

Those skilled in the art will recognize, or be able to ascertain using no more than routine experimentation, many equivalents to the specific embodiments of the invention described herein. Such equivalents are intended to be encompassed by the following claims.

1. A method for treating a disorder associated with a sleep spindle deficit, the method comprising administering to a subject in need thereof an effective amount of a group II metabotropic glutamate receptor modulator.

2. The method of claim 1, wherein the group II metabotropic glutamate receptor modulator is a mGluR_{2/3} agonist.

3. The method of claim 2, wherein the mGluR_{2/3} agonist is selected from the group consisting of LY354740, MGS0028, LY379268, LY2934747, LY2969822, LY404040, LY404039, and LY2140023.

4. The method of claim 3, wherein the mGluR_{2/3} agonist is LY379268.

5. The method of claim 3, wherein the mGluR_{2/3} agonist is LY404039.

6. The method of claim 1, wherein the group II metabotropic glutamate receptor modulator is an mGlu₃-specific modulator.

7. The method of claim 6, wherein the mGlu₃-specific modulator is a mGlu₃ agonist or a mGlu₃ positive allosteric modulator (PAM).

8. The method of claim 7, wherein the mGlu₃ PAM is selected from the group consisting of DT011088, Mavalon-63 PAM, and Mavalon-207 PAM.

9. The method of claim 8, wherein the mGlu₃ PAM is Mavalon-63 PAM.

10. The method of any one of claims 1-9, wherein the subject is a human subject having, suspected of having, or at risk of developing a schizophrenia disorder.

11. The method of any one of claims 1-10, wherein the subject is a human subject having a mutation in the gene encoding the CACNA1I protein.

12. The method of any one of claims 1-11 further comprising administering to the subject an additional therapeutic agent.

13. The method of claim 12, wherein the additional therapeutic agent is selected from the group consisting of aripiprazole, asenapine, brexpiprazole, buspirone, cariprazine, chlorpromazine hydrochloride, clozapine, haloperidol, iloperidone, loxapine, lumateperone, lurasidone hydrochloride, molindone hydrochloride, olanzapine, paliperidone, perphenazine, prochlorperazine, quetiapine, risperidone, thiothixene, trifluoperazine, and ziprasidone.

14. A method for treating a disorder associated with a sleep spindle deficit, comprising administering an effective amount of a group II metabotropic glutamate receptor modulator to a subject who has been diagnosed with a disorder associated with a sleep spindle deficit.

15. A method for treating a disorder associated with a sleep spindle deficit, comprising:
determining that a subject has, is suspected of having, or is at risk of developing a disorder associated with a sleep spindle deficit, and
administering an effective amount of a group II metabotropic glutamate receptor modulator to the subject having, suspected of having, or at risk of developing, the disorder associated with the sleep spindle deficit.

16. The method of claim 14 or 15, wherein the group II metabotropic glutamate receptor modulator is a mGlu_{2/3} agonist.

17. The method of claim 16, wherein the mGlu_{2/3} agonist selected from the group consisting of LY354740, MGS0028, LY379268, L12934747, LY2969822, LY404040, LY404039, and LY2140023.

18. The method of claim 17, wherein the mGluR_{2/3} agonist is LY379268.

19. The method of claim 17, wherein the mGluR_{2/3} agonist is LY404039.

20. The method of claim 14 or 15, wherein the group II metabotropic glutamate receptor modulator is an mGlu₃-specific modulator.

21. The method of claim 20, wherein the mGlu₃-specific modulator is a mGlu₃ agonist or a mGlu₃ positive allosteric modulator (PAM).

22. The method of claim 21, wherein the mGlu₃ PAM is selected from the group consisting of DT011088, Mavalon-63 PAM, and Mavalon-207 PAM.

23. The method of claim 22, wherein the mGlu₃ s Mavalon-63 PAM.

24. The method of any one of claims 14-23, wherein the subject is a human subject.

25. The method of claim 15, wherein determining that a subject has, is suspected of having, or is at risk of developing a disorder associated with a sleep spindle deficit comprises detecting sleep spindles with an electroencephalogram (EEG).

26. The method of claim 15, wherein determining that a subject has, is suspected of having, or is at risk of developing a disorder associated with a sleep spindle deficit comprises determining whether the subject has a mutation in the gene encoding the CACNA1I protein.

27. The method of claim 26, wherein the mutation comprises a mutation at the amino acid residue corresponding to amino acid residue 1346 in the CACNA1I protein depicted by SEQ ID NO: 1.

28. The method of claim 27, wherein the mutation at the amino acid residue corresponding to amino acid residue 1346 in the CACNA1I protein depicted by SEQ ID NO: 1 is a substitution of R1346H.

29. The method of any one of claims 14-28, wherein the disorder associated with the sleep spindle deficit is associated with a schizophrenia disorder in the subject.

30. A method for treating a disorder associated with a sleep spindle deficit in a subject, the method comprising:
administering an effective amount of a group II metabotropic glutamate receptor modulator to a subject that has a mutation at the amino acid residue corresponding to amino acid residue 1346 in the CACNA1I protein depicted by SEQ ID NO: 1.

31. A method for treating a CACNA1I gene disorder in a subject, the method comprising:
determining whether a subject has a mutation at the amino acid residue corresponding to amino acid residue 1346 in the CACNA1I protein depicted by SEQ ID NO: 1, and administering an effective amount of a group II metabotropic glutamate receptor modulator to the subject if the subject has a mutation at the amino acid residue corresponding to amino acid residue 1346 in the CACNA1I protein depicted by SEQ ID NO: 1.

32. The method of claim 30 or 31, wherein the group II metabotropic glutamate receptor modulator is a mGlu_{2/3} agonist.

33. The method of claim 32, wherein the mGlu_{2/3} agonist selected from the group consisting of LY354740, MGS0028, LY379268, LY2934747, LY2969822, LY404040, LY404039, and LY2140023.

34. The method of claim 30 or 31, wherein the group II metabotropic glutamate receptor modulator is an mGlu₃-specific modulator.

35. The method of claim 34, wherein the mGlu₃-specific modulator is a mGlu₃ agonist or a mGlu₃ positive allosteric modulator (PAM).

36. The method of claim 35, wherein the mGlu₃ PAM is selected from the group consisting of DT011088, Mavalon-63 PAM, and Mavalon-207 PAM.

37. The method of claim 36, wherein the mGlu₃ PAM is Mavalon-63 PAM.

38. The method of any one of claims 30-37, wherein the subject is a human subject.

39. The method of claim 30 or 31, wherein the mutation at the amino acid residue corresponding to amino acid residue 1346 in the CACNA1I protein depicted by SEQ ID NO: 1 is a substitution of arginine to histidine.

40. The method of any one of claims 31-39, wherein the CACNA1I gene disorder is associated with a sleep spindle deficit.

41. The method of claim 40, wherein the disorder associated with the sleep spindle deficit is a schizophrenia disorder.

42. The method of any one of claims 30-40, wherein the disorder is bipolar disorder, intellectual disability, schizophrenia disorders, epilepsy, or an autism spectrum disorder.

43. The method of claim 41 or 42, wherein the schizophrenia disorder is selected from the group consisting of paranoid schizophrenia, disorganized schizophrenia, catatonic schizophrenia, childhood schizophrenia, and schizoaffective disorder.

44. The method of any one of claims 30-43 further comprising administering to the subject an additional therapeutic agent.

45. The method of claim 44, wherein the additional therapeutic agent is selected from the group consisting of aripiprazole, asenapine, brexpiprazole, buspirone, cariprazine, chlorpromazine hydrochloride, clozapine, haloperidol, iloperidone, loxapine, lumateperone, lurasidone hydrochloride, molindone hydrochloride, olanzapine, paliperidone, perphenazine, prochlorperazine, quetiapine, risperidone, thiothixene, trifluoperazine, ziprasidone, antipsychotics including aripiprazole, asenapine, cariprazine, loxapine, lurasidone hydrochloride, olanzapine, olanzapine and fluoxetine, quetiapine, risperidone, ziprasidone, clozapine, paliperidone, cariprazine, lurasidone, haloperidol, and chlorpromazine, antidepressants including fluoxetine, SSRIs including citalopram, escitalopram, paroxetine, and sertraline, SNRIs including desyenlafaxine, duloxetine, and venlafaxine, tricyclics including amitriptyline, desipramine, imipramine, and nortriptyline, and MAOIs including phenelzine and tranylcypromine, anticonvulsants including carbamazepine, divalproex sodium, lamotrigine, valproate sodium, valproic acid, and topiramate, mood stabilizers including lithium and lithium carbonate, benzodiazepines including lorazepam, clonazepam, diazepam, alprazolam, and chlordiazepoxide, metformin, memantine, flumazenil, or meclofenoxate, risperidone, lithium carbonate, methylphenidate, procyclidine, ferrous fumarate+vitamins+lactulose+cod liver oil+various skin ointments, clobazam+lorazepam, rectal diazepam+buccal midazolam, ethosuximide, felbamate, gabapentin, gabapentin and lidocaine, gabapentin and lidocaine and prilocaine, lacosamide, levertiracetatn, oxcarbazepine, peratnpanel, topiramate, vaiproate, zonisamide, cannabidiol, cenobamate, phenytoin, ezogabine, rufinamide, stiripentol, vigabatrin, eslicarbazepine acetate, pregabalin, and tiagabine, midazolam, clobazam, barbiturates including phenobarbital and primidone, stimulants including methylphenidate, or alpha-2-adrenergic agonists including clonidine, and guanfacine.


SEQUENCE LISTING (2 sequences)

SEQ ID NO: 1 - protein, 2223 aa, Homo Sapiens
MAESASPPSSSAAAPAAEPGVTTEQPGPRSPPSSPPGLEEPLDGADPHVPHPDLAPIAFFCLRQTTSPRNWCIKMVCNPWFECVSMLVILLNCVTLGMYQPCDDMDCLSDRCKILQVFDDFIFIFFAMEMVLKMVALGIFGKKCYLGDTWNRLDFFIVMAGMVEYSLDLQNINLSAIRTVRVLRPLKAINRVPSMRILVNLLLDTLPMLGNVLLLCFFVFFIFGIIGVQLWAGLLRNRCFLEENFTIQGDVALPPYYQPEEDDEMPFICSLSGDNGIMGCHEIPPLKEQGRECCLSKDDVYDFGAGRQDLNASGLCVNWNRYYNVCRTGSANPHKGAINFDNIGYAWIVIFQVITLEGWVEIMYYVMDAHSFYNFIYFILLIIVGSFFMINLCLVVIATQFSETKQREHRLMLEQRQRYLSSSTVASYAEPGDCYEEIFQYVCHILRKAKRRALGLYQALQSRRQALGPEAPAPAKPGPHAKEPRHYHGKTKGQGDEGRHLGSRHCQTLHGPASPGNDHSGRELCPQHSPLDATPHTLVQPIPATLASDPASCPCCQHEDGRRPSGLGSTDSGQEGSGSGSSAGGEDEADGDGARSSEDGASSELGKEEEEEEQADGAVWLCGDVWRETRAKLRGIVDSKYFNRGIMMAILVNTVSMGIEHHEQPEELTNILEICNVVFTSMFALEMILKLAAFGLFDYLRNPYNIFDSIIVIISIWEIVGQADGGLSVLRTFRLLRVLKLVRFMPALRRQLVVLMKTMDNVATFCMLLMLFIFIFSILGMHIFGCKFSLRTDTGDTVPDRKNFDSLLWAIVTVFQILTQEDWNVVLYNGMASTSPWASLYFVALMTFGNYVLFNLLVAILVEGFQAEGDANRSYSDEDQSSSNIEEFDKLQEGLDSSGDPKLCPIPMTPNGHLDPSLPLGGHLGPAGAAGPAPRLSLQPDPMLVALGSRKSSVMSLGRMSYDQRSLSSSRSSYYGPWGRSAAWASRRSSWNSLKHKPPSAEHESLLSAERGGGARVCEVAADEGPPRAAPLHTPHAHHIHHGPHLAHRHRHHRRTLSLDNRDSVDLAELVPAVGAHPRAAWRAAGPAPGHEDCNGRMPSIAKDVFTKMGDRGDRGEDEEEIDYTLCFRVRKMIDVYKPDWCEVREDWSVYLFSPENRFRVLCQTIIAHKLFDYVVLAFIFLNCITIALERPQIEAGSTERIFLTVSNYIFTAIFVGEMTLKVVSLGLYFGEQAYLRSSWNVLDGFLVFVSIIDIVVSLASAGGAKILGVLRVLRLLRTLRPLRVISRAPGLKLVVETLISSLKPIGNIVLICCAFFIIFGILGVQLFKGKFYHCLGVDTRNITNRSDCMAANYRWVHHKYNFDNLGQALMSLFVLASKDGWVNIMYNGLDAVAVDQQPVTNHNPWMLLYFISFLLIVSFFVLNMFVGVVVENFHKCRQHQEAEEARRREEKRLRRLEKKRRKAQRLPYYATYCHTRLLIHSMCTSHYLDIFITFIICLNVVTMSLEHYNQPTSLETALKYCNYMFTTVFVLEAVLKLVAFGLRRFFKDRWNQLDLAIVLLSVMGITLEEIEINAALPINPTIIRIMRVLRIARVLKLLKMATGMRALLDTVVQALPQVGNLGLLFMLLFFIYAALGVELFGKLVCNDENPCEGMSRHATFENFGMAFLTLFQVSTGDNWNGIMKDTLRDCTHDERSCLSSLQFVSPLYFVSFVLTAQFVLINVVVAVLMKHLDDSNKEAQEDAEMDAELELEMAHGLGPGPRLPTGSPGAPGRGPGGAGGGGDTEGGLCRRCYSPAQENLWLDSVSLIIKDSLEGELTIIDNLSGSIFHHYSSPAGCKKCHHDKQEVQLAETEAFSLNSDRSSSILLGDDLSLEDPTACPPGRKDSKGELDPPEPMRVGDLGECFFPLSSTAVSPDPENFLCEMEEIPFNPVRSWLKHDSSQAPPSPFSPDASSPLLPMPAEFFHPAVSASQKGPEKGTGTGTLPKIALQGSWASLRSPRVNCTLLRQATGSDTSLDASPSSSAGSLQTTLEDSLTLSDSPRRALGPPAPAPGPRAGLSPAARRRLSLRGRGLFSLRGLRAHQRSHSSGGSTSPGCTHHDSMDPSDEEGRGGAGGGGAGSEHSETLSSLSLTSLFCPPPPPPAPGLTPARKFSSTSSLAAPGRPHAAALAHGLARSPSWAADRSKDPPGRAPLPMGLGPLAPPPQPLPGELEPGDAASKRKR

SEQ ID NO: 2 - protein, 2199 aa, Mus musculus
MADSNLPPSSSAAPDPEPGITEQPGPRSPPPSPPGLEEPLDGTNPDVPHPDLAPVAFFCLRQTTSPRNWCIKMVCNPWFECVSMLVILLNCVTLGMYQPCDDMECLSDRCKILQVFDDFIFIFFAMEMVLKMVALGIFGKKCYLGDTWNRLDFFIVMAGMVEYSLDLQNINLSAIRTVRVLRPLKAINRVPSMRILVNLLLDTLPMLGNVLLLCFFVFFIFGIIGVQLWAGLLRNRCFLEENFTIQGDVALPPYYQPEEDDEMPFICSLSGDNGIMGCHEIPPLKEQGRECCLSKDDMYDFGAGRQDLNASGLCVNWNRYYNVCRTGNANPHKGAINFDNIGYAWIVIFQVITLEGWVEIMYYVMDAHSFYNFIYFILLIIVGSFFMINLCLVVIATQFSETKQREHRLMLEQRQRYLSSSTVASYAEPGDCYEEIFQYVCHILRKAKRRALGLYQALQNRRQATGPGTPAPAKPGPHAKEPSHCKLCPRHSPLDTTPHTLVQPISAILASDPSSCPRCQHEAGRRPSGLGSTDSGQEGSGSGGSAEAEANGDGPQSSEDGVSSGLGKEEEQEDGAARLCGDVWRETRAKLRGIVDSKYFNRGIMMAILVNTVSMGIEHHEQPEELTNILEICNVVFTSMFALEMILKLAAFGLFDYLRNPYNIFDSIIVIISIWEIVGQADGGLSVLRTFRLLRVLKLVRFMPALRRQLVVLMKTMDNVATFCMLLMLFIFIFSILGMHIFGCKFSLRTDTGDTVPDRKNFDSLLWAIVTVFQILTQEDWNVVLYNGMASTTPWASLYFVALMTFGNYVLFNLLVAILVEGFQAEGDANRSYSDEDQSSSNLEELDKLPEGLDSSRDLKLCPIPMTPNGHLDPSLPLGGHLGPAGAMGAAPRLSLQPDPVLVALESRKSSVMSLGRMSYDQRSLSSSRSSYYGPWGRSGTWASRRSSWNSLKHKPPSAEHESLLSGERGGSCVRACEGAREDAPPRAAPLHAPHTHHAHHGPHLAHRHRHHRRTLSLDTRDSVDLAELVPVVGAHSRAAWRAAGQAPGHEDCNGRMPNIAKDVFTKMDDRRDRGEDEEEIDYTLCFRVRKMIDVYKPDWCEVREDWSVYLFSPENKFRILCQTIIAHKLFDYVVLAFIFLNCITIALERPQIEAGSTERIFLTVSNYIFTAIFVGEMTLKVVSLGLYFGEQAYLRSSWNVLDGFLVFVSIIDIVVSVASAGGAKILGVLRVLRLLRTLRPLRVISRAPGLKLVVETLISSLKPIGNIVLICCAFFIIFGILGVQLFKGKFYHCLGVDTRNITNRSDCVAANYRWVHHKYNFDNLGQALMSLFVLASKDGWVNIMYNGLDAVAVDQQPVTNHNPWMLLYFISFLLIVSFFVLNMFVGVVVENFHKCRQHQEAEEARRREEKRLRRLEKKRRKAQRLPYYATYCPTRLLIHSMCTSHYLDIFITFIICLNVVTMSLEHYNQPTSLETALKYCNYMFTTVFVLEAVLKLVAFGLRRFFKDRWNQLDLAIVLLSVMGITLEEIEINAALPINPTIIRIMRVLRIARVLKLLKMATGMRALLDTVVQALPQVGNLGLLFMLLFFIYAALGVELFGKLVCNDENPCEGMSRHATFENFGMAFLTLFQVSTGDNWNGIMKDTLRDCTHDERSCLSSLQFVSPLYFVSFVLTAQFVLINVVVAVLMKHLDDSNKEAQEDAEMDAEIELEMAHGLGPGPGPCPCPCPCPCPCPCPGPRMPTSSPGAPGRGSGGAGVGGDTESHLCRHCYSPAQETLWLDSVSLIIKDSLEGELTIIDNLSGSIFHHYSSPAGCDKCHHDKQEVQLAETEAFSLNSDRSSSVLLGDDLSLEDPTACPQGPKESKGELEPPEPMQAGDLDECFFPFAGEPVSAGPESLLCEMGAIPFNPVQSWLKHESNQAPPSPFSPDGSSPLLQMPAEFFHPAVSASQKGQEPGMSSGTLPKIALQGSWASLRSPSVNCTLLRQATVSDTSLDASPSSSAGSLQTTLEDSLTLSDSPRRALGPPVQVPGPRASLSPATRRRLSLRGRGLFSLRGLRAHQRSHSSGGSTSPGCTYHDSMDPSDEEGRGGAGGGGAGSEHSETLSSLSLTSLFCLPPTLPPPGLTPARKFSSTSSLAAGPGRPGATVSVRGLARSPSWAADRSKDPPGQAQLASGFGSSAPEPQPPPGESTDAASKRKR